(19) United States
(12) Patent Application Publication
(10) Pub. No.: US 2021/0302436 A1
(43) Pub. Date: Sep. 30, 2021

(54) Method, Systems, and a Kit for Detection, Diagnosis, Monitoring and Treatment of COVID-19

(71) Applicant: Baseline Viral Diagnostics, Inc., Irvine, CA (US)

(72) Inventors: Balwant Rai, Dragor (DK); Jasdeep Kaur, Dragor (DK)

(73) Assignee: Baseline Viral Diagnostics, Inc., Irvine, CA (US)

(21) Appl. No.: 17/075,470

(22) Filed: Oct. 20, 2020

Related U.S. Application Data
(63) Continuation of application No. PCT/US2020/040701, filed on Jul. 2, 2020.

(30) Foreign Application Priority Data
Mar. 23, 2020 (IN) 202011012622

(51) Int. Cl. G01N 33/68; G01N 33/569; G01N 33/543; G01N 21/64

(52) U.S. Cl. CPC G01N 33/6893; G01N 33/56983; G01N 33/54306; G01N 2333/61; G01N 2333/5412; G01N 2333/5428; G01N 2333/57; G01N 21/6428

(57) ABSTRACT

Methods, systems, and kits for detection, diagnosis, monitoring, and/or treatment of viral infections such as represented by the COVID-19 disease are described. The methods, systems, and kits are capable of detection of salivary biomarkers which correlate with, and are indicative of, COVID-19 in a subject. Detection of the biomarkers in a saliva sample provides opportunities for a COVID-19 or other viral detection assay which is non-invasive, produces rapid results, and can be implemented in the field on a wide geographic basis for individualized screening or mass screenings for COVID-19 or other viral infections.

### RELATED APPLICATIONS

This application is a continuation of international patent application Serial No. PCT/US2020/040701 filed with the United States Patent & Trademark Office as PCT Receiving Office on Jul. 2, 2020 and designating the United States which claims the benefit and priority date of Indian patent application Serial No. 202011012622 filed with the Indian Patent Office on Mar. 23, 2020. The entire contents of both international patent application Serial No. PCT/US2020/040701 and Indian patent application Serial No. 202011012622 are incorporated herein by reference for continuity.

### FIELD

The present invention relates to the detection of biomarkers. More specifically, the invention relates to the detection of biomarkers for the diagnosis, prognosis, monitoring, treatment, and management of viral infections in a subject.

### BACKGROUND

Severe acute respiratory syndrome coronavirus 2 (SARS-CoV-2) is the strain of virus responsible for causing the human respiratory illness referred to as the Coronavirus Disease 2019, abbreviated COVID-19. Colloquially known by the term “Coronavirus”, the SARS-CoV-2 virus was previously referred to by its provisional name, 2019 novel coronavirus (2019-nCoV), and has also been called human coronavirus 2019 (HCoV-19 or hCoV-19). SARS-CoV-2 is a positive-sense single-stranded RNA virus. It is contagious in humans.

The December 2019 outbreak of the novel SARS-CoV-2 virus has been classified as an international public health emergency by the World Health Organization (WHO) and has triggered a strong response by senior health officials worldwide. World Health Organization, Statement on the Second Meeting of the International Health Regulations (2005) Emergency Committee Regarding the Outbreak of Novel Coronavirus (2019-nCoV) (2020). The outbreak of the SARS-CoV-2 virus late in 2019 was first detected in Wuhan, Hubei Province, China where clusters of an acute respiratory illness were identified. Michelle L. Holshue, et al., First Case of 2019 Novel Coronavirus in the United States, New Eng. J. Med. (2020). The disease which began in Wuhan spread rapidly throughout the world. Camilla Rothe, et al., Transmission of COVID-19 Infection from an Asymptomatic Contact in Germany, New Eng. J. Med. (2020).

Through genomic sequencing and phylogenic analysis it has been determined that the novel SARS-CoV-2 virus is representative of a clade related to betacoronavirus-type human Severe Acute Respiratory Syndrome (SARS) and Middle East Respiratory Syndrome (MERS). David S. Hui, et al., The Continuing 2019-nCoV Epidemic Threat of Novel Coronaviruses to Global Health—The Latest 2019 Novel Coronavirus Outbreak in Wuhan, China, Int. J. Infect. Dis. 264-266 (2020); J S Eden, et al., An Emergent Clade of SARS-CoV-2 Linked to Returned Travelers from Iran, Virus Evol. (2020). SARS-CoV-2 also has close similarity to bat coronaviruses. Lan T. Phan, et al., Importation and Human-to-Human Transmission of a Novel Coronavirus in Vietnam, New Eng. J. Med. 382; 9 (2020).

Human symptoms of a SARS-CoV-2 viral infection are nonproductive cough, fever, dyspnea, myalgia, fatigue, radiographic evidence of pneumonia, as well as normal or decreased leukocyte counts. Nanshan Chen, et al., Epidemiological and clinical Characteristics of 99 Cases of 2019 Novel Coronavirus Pneumonia in Wuhan, China: A Descriptive Study, Lancet vol. 395 507-513 (2020). At present, there are no effective therapies or vaccines for treatment of this infectious disease.

Strategies exist for detection of COVID-19 in a human subject and for monitoring progress of the disease. The most commonly used laboratory diagnostic tests to detect COVID-19 disease in a subject are PCR-based genomic tests and SARS-CoV-2 antibody serological tests. Chaolin Huang, et al., Clinical Features of Patients Infected with 2019 Novel Coronavirus in Wuhan, China, Lancet vol. 395 496-307 (2020). Progress of COVID-19 disease in a human subject can be followed and monitored by serial chest radiography. Chen Lei, et al., Analysis of Clinical Characteristics of 29 Cases of 2019-nCoV Pneumonia, Chinese Journal of Tuberculosis and Respiration 43 (2020). Serial chest radiography is effective, but only after it is known that the subject is infected with SARS-CoV-2 and has the COVID-19 disease.

While effective, existent genomic and antibody-based assays for COVID-19 disease have certain disadvantages. Such disadvantages could render the genomic and antibody-based assays sub-optimal for use in certain important applications.

Two types of genomic assays capable of detecting viral genomic nucleic acid sequences indicative of a SARS-CoV-2 infection are real-time polymerase chain reaction (RT-PCR) and polymerase chain reaction (PCR). While accurate at detecting the existence of viral nucleic acid in a subject, these genomic assays have certain disadvantages. One disadvantage is the invasiveness associated with collection of the tissue and fluid samples required for the analysis. For example, it may be necessary to insert a swab into the sinus region of a subject to obtain the necessary mucus sample. Trained medical personnel are required to collect the sample. The sample collection process can be uncomfortable to the subject.

A further disadvantage of PCR-based assays is that extensive physical infrastructure and human resources are required to perform the genomic assays on the collected samples. Processing of the collected COVID-19 samples by means of PCR-based assays requires specialized biocontainment laboratories operated by highly-trained technicians. These laboratories are usually located within medium to large hospitals or research facilities. The COVID-19 pandemic is placing severe workload demands on these laboratories. In addition, a relatively long 2 to 3 hours is required to produce a result using PCR-based assays. These requirements render PCR-based assays unsatisfactory for use in the field (i.e., where needed) for rapid detection of COVID-19 in a subject or a group of subjects.

A further disadvantage of genomic assays is that the viral genome may not be detectable once the subject recovers from an initial viral infection. Therefore, a genomic assay may be ineffective at detection of prior exposure to SARS-CoV-2 in a subject who has recovered from COVID-19.

Antibody-based assays exist in recognition that antibodies are produced as an innate and/or adaptive immune response induced by a viral infection and detection of the antibodies indicates existence of the viral infection. It has been reported that the existence of Immunoglobulin M (IgM) and Immunoglobulin G (IgG) in blood serum has the potential to be indicative of COVID-19 in a subject. Zhengtu Li, et al., Development and Clinical Application of a Rapid IgM-IgG Combined Antibody Test for SARS-CoV-2 Infection Diagnosis, J. Med. Virol. 1-7 (2020).

An important disadvantage associated with serological-based antibody detection is the potential of false negative results. False negative results can occur because the antibody load in blood serum can increase or decrease over time such that an assay performed during a period of decreased antibody load could result in a false negative indication of the existence of a viral infection.

A further disadvantage of any serum-based antibody assay is that the process of drawing venous blood or drawing blood by finger stick is highly invasive. Given the invasive nature of serological tests and the current need to massively increase testing responsive to COVID-19, the US Food and Drug Administration has been granting Emergency Use Authorization (EUA) for testing modalities, including serological lateral immunoassays for COVID-19. It is important for physicians to understand that most assay products marketed in this category currently require a Clinical Laboratory Improvement Amendments laboratory certification for moderate- or high-complexity tests to be performed. Accordingly, and despite their simplicity, most serological lateral immunoassays for COVID-19 cannot be performed in a typical private office setting. FDA CDRH (2020). (Document contains nonbinding recommendations and supersedes “Policy for Diagnostics Testing in Laboratories Certified to Perform High-Complexity Testing under Clinical Laboratory Improvement Amendments (CLIA) prior to Emergency Use Preface Public Comment.”)

In summary, diagnosis of COVID-19 is presently based on laboratory, clinical history, and chest radiographic findings, but verification of COVID-19 in a subject currently relies on nucleic acid-based assays. The nucleic acid-based assays such as RT-PCR and PCR are effective to verify the diagnosis of COVID-19 or other viral infections, but they are relatively slow to complete and are relatively complex and expensive. They are not suitable for use in the field or where a rapid result is required. Antibody-based assays for COVID-19 may be inaccurate and invasive.

Accordingly, it would be an improvement in the art to provide an improved assay for detection of the COVID-19 disease and other viral infections which would enable early detection, diagnosis, monitoring, and treatment of the disease, which would be capable of detecting COVID-19 in a subject irrespective of whether the subject is asymptomatic or symptomatic of COVID-19, which would produce accurate and reliable results, which would be non-invasive, which would be rapid, which would be easy to administer, which could be administered in the field, which would be economical and therefore capable of widespread use for a large population of people, and which would generally provide an opportunity for better healthcare outcomes.

### SUMMARY

Described herein are methods, systems, and kits for detection, diagnosis, monitoring and treatment of the viral infection known as COVID-19, and potentially other types of viral infections. The inventions described herein are based on the recognition that certain salivary biomarkers are highly predictive of a SARS-CoV-2 viral infection in a subject and that detection of such biomarkers as described herein can be used to differentiate between healthy uninfected subjects and infected but asymptomatic subjects as well as to differentiate between different levels or categories of severity of COVID-19 in the subject. Detection of this biological information in a subject's saliva can, in turn, be used to diagnose, monitor, and treat COVID-19.

It has been found that salivary biomarkers yield particularly reliable results in detection of SARS-CoV-2 viral infections. Salivary biomarkers which correlate strongly with COVID-19 may be one or more of Immunoglobulin G1 (IgG1), Immunoglobulin G3 (IgG3), Immunoglobulin G4 (IgG4), total Immunoglobulin G (IgG), Immunoglobulin M (IgM), Immunoglobulin A (IgA), Interleukin 2 (IL-2), Interleukin 6 (IL-6), Interleukin 10 (IL-10), Interferon gamma (IFN-γ), Hepatocyte growth factor (HGF), Colony stimulating factor 1 (CSF-1), Interleukin 18 (IL-18), and D-dimer.

Besides yielding highly accurate biomarker information indicative of COVID-19, saliva as a biomarker source has other unique and compelling advantages. Advantages include noninvasiveness of collection, ease of analysis, fast and easily understood results, and low cost of administration providing opportunities for saliva-based methods, systems, and kits that can be implemented to detect COVID-19 for one or many subjects on a widespread basis. The ability to implement saliva-based detection of COVID-19 provides opportunities to screen large populations responsive to a global pandemic such as that presented by the COVID-19 disease.

In embodiments, a method of detecting biomarkers indicative of the SARS-CoV-2 virus in a human subject is provided. The method may include obtaining a saliva sample from the subject and detecting whether one or more biomarkers indicative of the SARS-CoV-2 virus is present in the saliva sample. Examples of salivary biomarkers which may be detected include the aforementioned IgG1, IgG3, IgG4, total IgG, IgM, IgA, IL-2, IL-6, IL-10, IFN-γ, HGF, CSF-1, IL-18, and D-dimer. Many types of assays may be implemented to detect the biomarker(s) of interest, examples of which include lateral flow immunochromatographic assays (LFA), and enzyme-linked immunosorbent assays (ELISA). In examples of these types of assays, agents, such as antibodies with affinity for a type of biomarker, are contacted by the saliva sample and bind with a specific one of the biomarkers. Such agent or antibodies may be secured to a solid support thus immobilizing the bound biomarkers. Detecting of the binding between the agent and the specific biomarkers yields a result positive for COVID-19. Based on the extent of detected binding, levels of severity of COVID-19 can be ascertained. The detection includes detecting the lack of, or insufficiency of, binding thus yielding a negative result indicative of a healthy non-infected subject. In embodiments, detecting can be accomplished by binding of labeled antibodies to immobilized salivary biomarkers providing a visible indication, such as a color change.

The agent or antibodies may provide a visible indication when the at least one biomarker in the saliva sample meets or exceeds a reference value or amount. The reference value or amount may be derived from healthy subjects not infected with the SARS-CoV-2 virus so that a visible indication is evidence that the subject is infected and should take precautions to avoid spread of the virus and/or should seek medical assistance.

Methods may include detecting combinations of salivary biomarkers which are highly predictive of COVID-19 and detection of such combinations provides information indicative of a Sars-CoV-2 infection. In embodiments, combinations may include IgG3 and IL-6, IgG3 and IgM, IgM and IL-6, IgG3 and CSF-1, IgG3 and HGF, IgG3 and D-dimer, IL-6 and CSF-1, CSF-1 and HGF, and HGF and D-dimer and the agents or antibodies may have affinity for such combinations. Detection of the combination of IgG3, IgM and IL-6 is highly indicative of the Sars-CoV-2 infection and such combination can be identified in an assay with a desirably small group of three different agents or antibodies. Further accuracy can be provided by additional implementation of agents or antibodies capable of binding with HGF. Detection of other biomarker combinations which yield results indicative of a Sars-CoV-2 infection include IgG3 and IgA and the combination of IgG3, IgA, and IgM. Methods and assays to detect yet other combinations may be implemented as described herein. In embodiments, a diagnosis of COVID-19 may be made if the detected amount of the biomarker or biomarkers meets or exceeds a reference value or amount indicative of the disease.

The invention may be implemented in the form of a kit and/or a system. In embodiments, a kit for detecting salivary biomarkers indicative of a SARS-CoV-2 viral infection in a subject may include an assay. The assay may have a solid support on which a plurality of agents have been affixed, directly or indirectly, and which bind to one or more biomarker in a saliva sample obtained from the subject. The solid support could be provided as part of an LFA, or an ELISA, or another type of assay. The agent or agents may have an affinity for one or more of the aforementioned IgG1, IgG3, IgG4, total IgG, IgM, IgA, IL-2, IL-6, IL-10, IFN-γ, HGF, CSF-1, IL-18, and D-dimer biomarkers and each agent may bind to a different single biomarker. Kits tailored to detect biomarker combinations such as those previously described may be implemented. In embodiments, the agent or agents may be antibodies. Additional labeled antibodies with an affinity for specific ones of the biomarkers may be utilized to enable formation of a visible complex if one or more of the biomarkers is present in the saliva sample.

Detection of the visible complex, such as by a color change yields a result positive for COVID-19 and the extent of detected binding enables levels of severity of COVID-19 to be determined. Detecting that a visible complex has not formed or has formed insufficiently provides a result indicative that the subject is healthy.

In embodiments, a system for detecting biomarkers indicative of SARS-CoV-2 virus in a saliva sample may include an assay with at least one binding agent specific to one or more biomarker according to the previously-described embodiments, a measurable label that indicates a proportional reaction based on the amount of biomarker present in the saliva sample, and a measurement device operable to utilize the label to provide a qualitative and/or quantitative measure of the one or more biomarker indicative of whether the subject is infected with the SARS-CoV-2 virus. Measurement devices which may be implemented to detect the amount of the label may include optical-type readers.

The invention may be implemented as part of a treatment program to ascertain the effectiveness of pharmaceutical agents in treating or lessening the symptoms of COVID-19 in a subject. Changes in the aforementioned one or more of IgG1, IgG3, IgG4, total IgG, IgM, IgA, IL-2, IL-6, IL-10, IFN-γ, HGF, CSF-1, IL-18, and D-dimer biomarkers responsive to a pharmaceutical agent may be utilized to determine efficacy of the treatment.

These and other embodiments and specific and possible advantages will become evident with reference to the following description.

### DETAILED DESCRIPTION

The present invention relates to improvements in the detection, diagnosis, monitoring, and treatment of SARS-CoV-2 viral infections responsible for the disease known as COVID-19 as well as potentially other types of viral infections. Methods, systems, and kits according to the invention may be implemented by means of saliva harvested from a human subject and include assaying of a saliva sample for the presence of one, more than one, or multiple different biomarker combinations which correlate strongly with the existence, severity, and progression of COVID-19 in the human subject. Certain assay embodiments may be implemented ex vivo in that they can occur apart from the subject.

The correlation of the salivary biomarkers with COVID-19 is strong in symptomatic subjects and, importantly, in asymptomatic subjects, providing a powerful tool by which to identify asymptomatic carriers and potential spreaders of the disease. Biomarker information may be further used to determine or estimate the effectiveness of a particular treatment in limiting and/or reversing progression of COVID-19 or other viral infections in a subject. The ability to implement the invention to obtain the aforementioned types of information by means of assaying saliva provides important opportunities for the accurate, rapid, non-invasive and inexpensive testing of one or many subjects for COVID-19 and COVID-19 severity.

Methods, systems, and kits according to the invention may be implemented in any location including at a hospital, a clinic, a laboratory, as well as in the “field” at a needed location apart from any medical or laboratory facility. For example, the invention may be implemented at a home, a school, a business, a testing node, a point of care, or even at public or private events for purposes of screening groups of people for the presence of COVID-19.

As described herein, it has been found that certain biomarkers, if present in saliva, have characteristics useful in the detection, diagnosis, monitoring, and treatment of COVID-19 infections and potentially other viral infections. In embodiments, one, two, or combinations of more than two of the biomarkers IgG1, IgG3, IgG4, Total IgG, IgM, IgA, IL-2, IL-6, IL-10, IFN-γ, HGF, CSF-1, IL-18, and D-dimer have been found to correlate strongly with the existence, severity, and progression of COVID-19 in a human subject. It is envisioned that other biomarkers having similar characteristics to those listed above, such as proteins, peptides, and genetic and transcriptomic organic and inorganic biomarkers in saliva may also have utility in detection of COVID-19 in a subject.

Methods, systems, and kits for assaying saliva samples according to the invention may be implemented in many different ways according to the needs of the medical professional, technician, care giver, and/or the infected subject. Modes of implementation may include, for example, assays such as lateral flow immunochromatographic assays (LFA) and enzyme-linked immunosorbent assays (ELISA), a ready-to-use assay device, a “lab-on-a-chip”, or even as a biosensor accessory for use with a mobile device such as an iOS-based iPhone or iPad or with an Android-based mobile device. Salivary biomarkers may be qualitatively or quantitatively measured using these and other assaying strategies for the detection, diagnosis, monitoring, and treatment of COVID-19 or other viral infections.

### Definitions

As used in this document, the singular forms “a”, “an”, and “the” include plural references unless the context clearly dictates otherwise.

“Assaying” means or refers to the analysis of a saliva sample to determine the presence of one or more salivary components, referred to herein as biomarkers. The assaying may be performed using many different processes in accordance with the subject matter disclosed herein. Non-limiting types of assays which may be implemented according to the invention include the aforementioned LFA and ELISA types of assays.

A “biomarker”, also known as a biological marker, means or refers to a measurable indicator of a biological state or condition. As described herein, examples of biomarkers which have been determined to be indicative of COVID-19 in a subject are IgG1, IgG3, IgG4, Total IgG, IgM, IgA, IL-2, IL-6, IL-10, IFN-γ, HGF, CSF-1, IL-18, and D-dimer.

A “biomarker panel” defines a set of biomarkers used alone, in combination, or in sub-combinations for the detection, diagnosis, prognosis, treatment, or monitoring of a disease or condition based on detection values for the set of biomarkers.

As used herein, the terms “comprising”, “including”, “containing”, “composition”, “consisting”, and “characterized by” are interchangeable, inclusive and open-ended and do not exclude additional methods or procedural steps.

“COVID-19” means or refers to the disease characterized by an infection with the SARS-CoV-2 virus, also known colloquially as “coronavirus”.

SARS-CoV-2, a source of COVID-19 disease, means or refers to severe acute respiratory syndrome coronavirus, including in forms with varying levels of severity. As used herein, a “confirmed” case of COVID-19 means or refers to a subject who has the SARS-CoV-2 virus as confirmed, for example, by a process such as real-time polymerase chain reaction (RT-PCR). The present invention is also useful to confirm the existence and severity of COVID-19 in a subject.

An “asymptomatic” case of COVID-19 means or refers to a subject who has a confirmed case of the disease but who lacks any relevant clinical symptoms of COVID-19 within the preceding 14 days before two consecutive negative RT-PCR results.

A “mild” case of COVID-19 means or refers to a subject with mild COVID-19 symptoms which cannot be classified as severe.

A “severe” case of COVID-19 means or refers to a subject showing any of the following severe symptoms associated with COVID-19: (1) shortness of breath, RR≥30 bpm, (2) blood oxygen saturation≤93% (at rest), PaO₂/FiO₂ ratio≤300 mmHg, and/or (3) pulmonary inflammation with a progression rate of greater than 50% within 24 to 48 hours.

“Detecting”, “measuring”, or “taking a measurement” define a qualitative or quantitative determination of the amount, or level, or concentration of a biomarker in the sample, including the absence of the biomarker. A measurement device operable to provide a qualitative or quantitative level of one or more biomarkers in the sample may be implemented.

“Er vivo” means or refers to experimentation or measurements done in an environment external to a subject.

As used herein, a “reference value” of a biomarker may be any of a relative value, an absolute value, a range of values, a value that has an upper and/or lower limit, an average value, a median value, a mean value, a value as compared to a control or baseline value, or a combination thereof. A reference value may also be articulated as a level or an amount or a concentration.

“Subject” or “individual” refer to a human being.

An “anti-SARS-CoV-2 antibody” means or refers to antibodies to the SARS-CoV-2 virus. In embodiments, anti-SARS-CoV-2 may be a human monoclonal or polyclonal antibody with an affinity for SARS-CoV-2. Such exemplary antibodies primarily target the trimeric spike (S) glycoproteins on the viral surface that mediate entry into host cells. The S protein has two functional subunits that mediate cell attachment (the S1 subunit, existing of four core domains S1A through S1D) and fusion of the viral and cellular membrane (the S2 subunit). Potent neutralizing antibodies often target the receptor interaction site in S1, disabling receptor interactions. It binds a conserved epitope on the spike SIB receptor-binding domain. Anti-SARS-CoV-2 antibodies may include, for example, IgM and/or IgG antibodies.

Colony stimulating factor 1 (CSF-1), also known as macrophage colony-stimulating factor (M-CSF), is a secreted cytokine which causes hematopoietic stem cells to differentiate into macrophages or other related cell types. Eukaryotic cells also produce M-CSF in order to combat intercellular viral infection.

D-dimer (also referred to as D dimer) is a fibrin degradation product (or FDP), a small protein fragment present in the blood after a blood clot is degraded by fibrinolysis. It is so named because it contains two D fragments of the fibrin protein joined by a cross-link.

Hepatocyte growth factor (HGF) or scatter factor (SF) is a paracrine cellular growth, motility and morphogenic factor. It is secreted by mesenchymal cells and targets and acts primarily upon epithelial cells and endothelial cells, but also acts on haemopoietic progenitor cells and T cells.

Immunoglobulin A (IgA) is abundant in serum, nasal mucus, saliva, breast milk, and intestinal fluid, accounting for 10 to 15% of human immunoglobulins. IgA forms dimers.

Immunoglobulin G (IgG) is the most profuse antibody isotype in the blood, accounting for 70 to 75% of human immunoglobulins (antibodies). IgG binds antigen and drives the recognition of antigen-antibody complexes by leukocytes and macrophages. IgG is transferred to the fetus through the placenta and protects the infant until its own immune system is functional. IgG is largely responsible for long-term immunity after infection or vaccination. Immunoglobulin G1, G3, and G4 are isotypes of IgG. Total IgG refers to total Immunoglobulin G in a saliva sample.

Immunoglobulin M (IgM) IgM usually circulates in the blood, accounting for about 10% of human immunoglobulins. IgM generally has a pentameric structure in which five basic Y-shaped molecules are linked together. B cells produce IgM first in response to microbial infection/antigen invasion. These are early phase immunoglobulins that will develop first during acute infection. Although IgM has a lower affinity for antigens than IgG, it has higher avidity for antigens because of its pentameric structure. IgM, by binding to the cell surface receptor, also activates cell signaling pathways.

Interferon gamma (IFN-γ), is a major immune-modulating molecule produced mainly by T-cells and natural killer cells activated by antigens, mitogens or alloantigens. Most immune cells express IFN-γ receptors and respond to IFN-γ-induced signaling by up-regulating MHC class I expression.

Interleukin 2 (IL-2) is a type of cytokine signaling molecule in the immune system. IL-2 is a protein that regulates the activities of white blood cells (leukocytes, often lymphocytes) that are responsible for immunity.

Interleukin 6 (IL-6) is an interleukin that acts as a pro-inflammatory cytokine. In humans, it is encoded by the IL6 gene.

Interleukin 10 (IL-10) is an anti-inflammatory cytokine. In humans, interleukin 10 is encoded by the IL10 gene.

Interleukin 18 (IL-18) is a protein which in humans is encoded by the IL18 gene. The protein encoded by this gene is a pro-inflammatory cytokine. IL-18 can modulate both innate and adaptive immunity and its dysregulation can cause autoimmune or inflammatory diseases.

The terminology used herein is for the purpose of describing specific embodiments only and is not intended to be limiting of the invention. Unless defined otherwise, all technical and scientific terms used herein have the same meanings as commonly understood by one of normative skill in the art.

### BACKGROUND

Coronaviruses represent a large family of viruses. Some of these types of viruses cause illness in humans while others cause illness in animals. Human coronaviruses are common and are typically linked to mild illnesses, similar to the common cold.

COVID-19, caused by the SARS-CoV-2 virus, is a new disease that had not previously been identified in humans and which is believed to have spread to humans from animal carriers. It is rare for an animal-based coronavirus to become infectious in human populations, and it is even more rare for such a coronavirus to be capable of spreading from person to person through close contact. There have been two other types of coronaviruses that have spread from animals to humans and which have caused severe illness in humans. These are severe acute respiratory syndrome coronavirus (SARS CoV) and Middle East respiratory syndrome coronavirus (MERS CoV).

As reported in the Journal of Medical Virology, coronaviruses are enveloped, nonsegmented, positive-sense single-stranded RNA virus genomes in the size ranging from 26 to 32 kilobases, the largest known viral RNA genome. The virion has a nucleocapsid composed of genomic RNA and phosphorylated nucleocapsid (N) protein, which is buried inside phospholipid bilayers and covered by two different types of spike proteins: the spike glycoprotein trimmer (S) that can be found in all CoVs, and the hemagglutinin-esterase (HE) that exists in some CoVs. The membrane (M) protein (a type III transmembrane glycoprotein) and the envelope (E) protein are located among the S proteins in the virus envelope. CoVs were given their name based on the characteristic crown-like appearance. Geng Li, et al., Coronavirus Infections and Immune Responses, J. Med. Virol. 424-432 (2020).

A SARS-CoV-2 infection in a human subject activates both innate and adaptive immune responses. Ordinarily, a rapid and well-coordinated immune response represents a potent first line of defense against a viral infection. But in the case of COVID-19 disease, an excessive inflammatory innate response and a dysregulated adaptive host immune defense actually cause harmful tissue damage at both the site of virus entry and at the systemic level. Initiation of an excessive innate immune response to SARS-CoV-2 virus in a subject can lead to the production of certain cytokines that induce a proinflammatory response and attract cells, such as neutrophils and macrophages, to the site(s) of infection and, in turn, cause damage to normal host tissues by releasing cytotoxic substances. Stanley Perlman, et al., Immunopathogenesis of Coronavirus Infections: Implications for SARS, Nat. Rev. Immunol. 917-927 (2005). In subjects suffering from COVID-19, the levels of some cytokines are significantly elevated, and cytokine storms can be associated with the severity of the disease. Y. Yang, et al., Plasma IP-10 and MCP-3 Levels are Highly Associated with Disease Severity and Predict the Progression of COVID-19, J. Allergy Clin. Immunol. (2020). The existence of these cytokine storms clearly reflects a widespread uncontrolled dysregulation of the host subject's immune response. The excessive pro-inflammatory host response has been theorized to induce an immune pathology resulting in the rapid course of acute lung injury and acute respiratory distress syndrome occurring in SARS-CoV-2 infected patients. C. Huang, et al., Clinical Features of Patients Infected with 2019 Novel Coronavirus in Wuhan, China, Lancet, 497-506 (2020); Z. Xu, et al., Pathological Findings of COVID-19 Associated with Acute Respiratory Distress Syndrome, Lancet Respir. Med. 8, 420-422 (2020).

The adaptive immune response to a SARS-CoV-2 infection is manifested by an antibody response between 10 to 21 days following infection. Detection of antibodies in mild cases of COVID-19 may require additional time (e.g., four weeks or more) and, in a small number of cases, antibodies in blood (i.e., IgM, IgG) are not detected at all (at least during the studies' time scale). Based on the currently available data, the IgM and IgG antibodies in blood responsive to SARS-CoV-2 develop between 6-15 days post disease onset. Juanjuan Zhao, et al., Antibody Responses to SARS-CoV-2 in Patients of Novel Coronavirus Disease 2019, medRxiv.org (2020).

Given the key role of the immune system in COVID-19, a deeper understanding of the mechanism behind the immune dysregulation, as well as of SARS-CoV-2 immune-escape mechanisms provides clues for the detection, diagnosis, monitoring, and treatment of SARS-CoV-2.

### Saliva as a Source of Biomarker Information

The present inventors have recognized that human saliva is a uniquely valuable source of repeatable biological information enabling detection, diagnosis, monitoring, and treatment of SARS-CoV-2 in a subject. Saliva refers to the watery liquid secreted into the mouth by glands. Saliva serves an aid in digestion and provides lubrication for chewing and swallowing. Saliva is such a valuable bodily fluid because it contains biomarkers indicative of the innate and adaptive immune reactions to infection by SARS-CoV-2 virus and potentially other viral infections. More specifically, saliva is a repository of cytokines and antibodies produced by the innate and adaptive immune reaction to SARS-CoV-2 and this biomarker information can be predictive of COVID-19 disease as well as the severity of the disease.

Saliva is a bodily fluid which is more stable than blood serum and, unlike blood, saliva is inclusive of all cytokines and antibodies produced in response to SARS-CoV-2. Blood samples can yield inconsistent types and amounts of immune reaction biomarkers creating or producing results which lack needed reproducibility. Blood-based biomarker analysis by different researchers using different methods has resulted in inconsistency in the types and concentration of proteins resulting in poor reproducibility of results. Factors contributing to the inconsistent results with blood-based biomarker analysis may include difficulties associated with blood sample preparation, with protein preparation, or with performance of the assays. In contrast, saliva samples produce specimens with repeatable types and amounts of immune reaction biomarkers.

Saliva samples may be easily harvested, or collected, from human subjects by means of what can be characterized as a “drooling” method. Saliva sample volumetric sizes of from about 1 ml to about 5 ml are sufficient for assaying according to aspects of the invention. Saliva harvesting may be stimulated or unstimulated. Stimulated saliva production can be achieved by insertion of a wand-like oral appliance into the subject's mouth followed by chewing or sucking on the appliance. Excess saliva is deposited into a tube, a vial, or another container. Unstimulated saliva production may involve relaxed drooling from the subject's lower lip into a tube, a vial, or another container. A 2% sodium azide solution may be added to each saliva sample to prevent microbial decomposition of the saliva.

Unstimulated saliva production may require about 10 to about 15 minutes to collect a 1 ml to about 5 ml of saliva sample depending on the subject. Typically, the subject is asked to rinse orally with water ten to fifteen minutes prior to collection of unstimulated saliva samples. Following collection, the saliva samples may be centrifuged at, for example, 1800 rpm for 5 minutes to remove debris. The centrifuged saliva samples may then be frozen or placed in an ice bed to await further analysis.

### Saliva-Based Assays

The present invention enables assaying of a saliva sample harvested or collected from a subject to determine whether the subject has been infected with SARS-CoV-2 virus, or, potentially, other viral infections. The information may be used to diagnose the subject as having COVID-19.

In general, a method according to the invention comprises the steps of (a) obtaining the saliva sample from the subject and (b) detecting the presence of the biomarkers indicative of the SARS-CoV-2 virus. Detecting may be carried out by assaying using various assays of the types described herein. In embodiments, salivary biomarkers which correlate with a SARS-CoV-2 viral infection are cytokines or antibodies. The cytokines or antibodies may be: Immunoglobulin G1 (IgG1), Immunoglobulin G3 (IgG3), Immunoglobulin G4 (IgG4), total Immunoglobulin G (IgG), Immunoglobulin M (IgM), Immunoglobulin A (IgA), Interleukin 2 (IL-2), Interleukin 6 (IL-6), Interleukin 10 (IL-10), Interferon gamma (IFN-γ), Hepatocyte growth factor (HGF), Colony stimulating factor 1 (CSF-1), Interleukin 18 (IL-18), and D-dimer. One, or a combination of two or more, of the aforementioned biomarkers in the saliva sample is indicative of the SARS-CoV-2 virus infection in the subject. A diagnosis of COVID-19 may be made based on the results.

Qualitative or quantitative measurement of the level or amount of the detected biomarkers may be conducted ex vivo of the subject utilizing the subject's saliva sample. In embodiments, the measured amount of the biomarker(s) can be compared to a reference value or amount (e.g. a concentration) of the biomarker(s) derived from subjects who are healthy (i.e., a control) and who are not infected with the SARS-CoV-2 virus. If the measured amount of biomarker exceeds the reference value or amount, that outcome would be indicative of a SARS-CoV-2 virus infection in the subject, whereas amounts below the reference value or amount would be indicative that the subject is not infected. This information could be particularly valuable in determining whether an asymptomatic subject is infected, or not infected, with the virus. A positive result is evidence that the subject is or may be infected and should take precautions to avoid spread of the virus and/or should seek medical assistance.

In embodiments, a qualitative measurement of the detected biomarker may include an assay in which a determination is made regarding whether the amount of biomarker in the saliva sample exceeds a reference value or amount or level, also referred to herein as a “cutoff” level. If the detected biomarker exceeds the cutoff level, that could trigger identification of a test line or lines on a lateral flow strip assay or change the color of a test pad in some visually-observable manner, thus providing a binary yes/no result indicative of infection or lack of infection.

In other embodiments, a quantitative measurement of detected biomarker may be conducted in which the strength of a color, fluorescence, or some other indicator can be used to quantify the amount of biomarker in the saliva sample. Quantitative measurement of detected biomarker may be used to determine whether the amount of biomarker exceeds the reference value or amount or level (i.e., the “cutoff” level) and may be useful to quantitatively determine the progression and severity of the infection (e.g., between asymptomatic, moderate, and severe states of infection). Quantitative changes in the amount or level or concentration of biomarkers in a subject's saliva when evaluated over a time period (e.g., hours, days, months, etc.) may further be used to determine or estimate the effectiveness of a particular treatment in limiting and/or reversing progression of a viral infection or other disorder.

### Examples of Methods, Systems, Kits, and Other Strategies

Methods according to the invention may be implemented with different types of assays. Examples of assays which can be implemented for purposes of detecting salivary biomarkers indicative of a SARS-CoV-2 viral infection may include, without limitation, (1) lateral flow immunochromatographic assays (LFA), (2) enzyme-linked immunosorbent assays (ELISA), (3) enzyme-linked fluorescence polarization immunoassays (FPIA), (4) homogeneous immunoassays, (5) quantitative point of-care tests using determination of chemiluminescence, fluorescence, magnetic particles, and latex agglutination, (6) gel electrophoresis, (7) gas chromatograph-mass spectrometry (GC-MS), (8) separation immunoassays, (9) heterogeneous immunoassays, (10) homogenous immunoassays, and (11) assays which will be developed in the future.

In aspects of the invention, a method for assaying a saliva sample may comprise an immunochromatographic assay (LFA), also referred to as a lateral flow assay or test. An LFA utilizes a pad along which a liquid sample, such as saliva, migrates by capillary action (i.e., wicking). The surface of the pad includes reactive molecules that show a visual positive or negative result indicative of whether the biomarker of interest is present in the saliva sample.

An LFA pad is based on a series of capillary beds, such as pieces of porous paper, microstructured polymer or sintered polymer. Each of these pads has the capacity to transport saliva spontaneously.

In a sandwich-type LFA, elements of an LFA strip may include, in a direction of flow, a sample loading pad, a conjugate pad, and an absorption pad. The sample loading pad is provided to receive and to hold an excess of the saliva or another fluid. The saliva flows to the conjugate pad from the sample loading pad. The conjugate pad is provided with antibodies specific to the salivary biomarker and which are labeled with a visual tag such as latex nanoparticles, gold nanoparticles, or some other detectable tracer. The antibodies bind to the biomarker as the saliva migrates through the conjugate pad marking the biomarker.

The conjugate pad may further include, in the direction of flow, at least one test line and a control line distal of the test line. Further test lines may be located between the test and control lines. Saliva including the biomarker antibody complex, if present, flows to a proximal test line where antibodies specific to another region of the biomarker are immobilized and bound to the conjugate pad substrate. An immobilized complex forms at the test line if the biomarker of interest is present in the saliva sample. A signal, typically a color, appears at the test line when the concentration or amount of biomarker is in excess of a cutoff level representative of a reference value, amount or level.

Additional test lines between the proximal test line and control line, if provided, may include antibodies specific to other biomarkers which may be in the saliva sample.

A distal control line beyond the test line or lines contains affinity ligands which bind to excess labeled antibodies carried to the control line by the saliva. The bound excess antibodies form an immobilized complex at the control line. A signal, typically a color, indicates that the sample has flowed through the test line or lines and that the labeled antibodies in the conjugate pad are active. The indication at the control line confirms that the assay is valid. Excess saliva flows past the test and control lines and to the absorption pad. Other LFAs, such as competitive-type assays, may be implemented.

The LFA strip may, for example, be housed within a cassette with a proximal end port provided to receive the saliva sample in contact with the sample application pad. A dilution buffer fluid may be provided for admixture with the saliva sample to modify the saliva viscosity. The test signal produced at the test and control lines of the strip may be produced within about 3 minutes to about 30 minutes following application of the subject's saliva to the sample loading pad on the corresponding lateral flow test strip.

An LFA for detection of SARS-Co-V-2 in a saliva sample may be implemented by means of a lateral flow test strip including a conjugate pad with one or more test line each comprising an immobilized anti-human-antibody specific for one of a human IgG1, IgG3, IgG4, Total IgG, IgM, IgA, IL-2, IL-6, IL-10, IFN-γ, HGF, CSF-1, IL-18, and D-dimer. The conjugate pad of such examples may include a labeled antibody specific to one of human IgG1, IgG3, IgG4, Total IgG, IgM, IgA, IL-2, IL-6, IL-10, IFN-γ, HGF, CSF-1, IL-18, and D-dimer.

If the target biomarker is present in the saliva sample, then a complex comprising the labeled antibody specific to the one of human IgG1, IgG3, IgG4, Total IgG, IgM, IgA, IL-2, IL-6, IL-10, IFN-γ, HGF, CSF-1, IL-18, and D-dimer forms with the immobilized antibody at the respective test line. If sufficient saliva is present in the assay, unbound labeled antibody forms a complex at the control line. As previously described, all complexes preferably provide a signal, such as a color, visible to the naked eye indicative that the selected biomarker is or is not present and that sufficient saliva was provided for a valid assay.

In embodiments, the biomarker amount or concentration in the saliva sample may be determined. In certain systems, a measurement device may be utilized to provide a qualitative and/or quantitative level of one or more biomarkers in the saliva sample. For example, a handheld lateral flow reader may be implemented to provide a quantitative assay result. An optical-type lateral flow reader may function in conjunction with a CCD or CMOS and may direct light energy at the test line at which the complex including labeled antibody indicative of the target biomarker is bound. The label can provide a proportional reaction based on the amount of biomarker present in the saliva sample. As an example, a reader may utilize a measure of fluorescence or color intensity from the label to determine concentration of the target biomarker. Using image processing technology, the lateral flow readers are capable of providing a qualitative or quantitative determination of the biomarker concentration.

In embodiments, LFA methods may be used to determine and to differentiate between early, intermediate, and late SARS-CoV-2 virus infection in a subject by detecting the presence and concentrations of one or more of IgG1, IgG3, IgG4, Total IgG, IgM, IgA, IL-2, IL-6, IL-10, IFN-γ, HGF, CSF-1, IL-18, and D-dimer.

Embodiments of the invention may be implemented in the form of a diagnostic kit purposed to assay a saliva sample and to detect one or more biomarkers indicative of SARS-CoV-2 in the saliva sample. Non-limiting types of assays which may be implemented in a kit according to the invention include LFA and ELISA types of assays of the types described herein.

Advantageously, embodiments of such a kit are capable of being provided in the form of a “ready-to-use” assay which is simple to use, rapid, and produces an easily understood result. Such a kit could be implemented at any location including at a hospital, a clinic, or a laboratory. Such a kit could further be utilized away from these types of institutional settings at a needed location. (i.e., in the “field”) For example, a kit according to the invention could be used at a home, a school, a business, a testing node, a point of care, or even at public or private events for purposes of screening groups of people for the presence of COVID-19. A “ready-to-use” assay could be portable, lightweight, inexpensive, and easy to use and therefore be capable of widespread use to potentially screen large groups of people.

One example of a kit that could be used to practice methods according to the present invention as a “ready-to-use” device is described in International Patent Application Serial No. PCT/US2020/033365 entitled Assay Device, System, Method, and Kit, the contents of which are incorporated herein by reference in their entirety. Embodiments of an assay device according to Application Serial No. PCT/US2020/033365 are well-suited for use in LFA-type assays and enable a saliva sample to be readily collected from a subject and then brought into contact with one or more LFAs contained in a sanitary housing to produce an assay result viewable through a window in the housing.

In embodiments, the result is complete within just a matter of minutes. The result could be as simple as a color change viewable on the LFA strip or in the form of a color band which forms at one or more test and control lines. The existence of a color change or of a color at the test line(s) may represent a “positive” result while the lack of a color change or of a color at the test line(s) could be a “negative” result. An on-board optical source may be used to assist the user in visualizing the test lines and control line and result provided at those test lines.

In other embodiments, a diagnostic kit capable of performing assaying methods for detection of one or more biomarkers indicative of a SARS-CoV-2 infection in a saliva sample could comprise an ELISA-type assay. ELISA assays are well suited for detection of salivary biomarkers such as antibodies, cytokines, peptides, or other molecule types.

In one form of an ELISA assay, saliva containing one or more of IgG1, IgG3, IgG4, Total IgG, IgM, IgA, IL-2, IL-6, IL-10, IFN-γ, HGF, CSF-1, IL-18, and/or D-dimer may be attached to a substrate such as a microtiter plate. An antibody specific to the biomarker may be applied over the surface so it can bind the biomarker. Such an antibody could be considered indirectly secured to the substrate provided by the microtiter plate. This antibody is linked to an enzyme and then any unbound antibodies are removed. In a final step, a substance containing the enzyme's substrate is added. If the biomarker is present in the saliva sample, there is binding and the subsequent reaction produces a detectable signal, most commonly a color change. The concentration of the biomarker can be quantified using a cutoff level or a reference value or amount.

In a sandwich form of ELISA capable of use in kit form, select antibodies with an affinity for a type of target biomarker can be linked to the substrate of the microtiter plate. Such an antibody could be considered directly secured to the substrate provided by the microtiter plate. Saliva containing biomarkers may be applied over the substrate. Following rinsing, a fluid with labeled antibodies with affinity for the target biomarker is applied over the substrate. The label can elicit a signal when a signaling reagent is applied. The signaling reagent is capable of providing a measurable signal proportional to the concentration of the target biomarker present on the substrate.

The kit may include instructions describing how to use the kit. In embodiments the instructions should explain steps such as how to collect a saliva sample, how much saliva is necessary, how to load the saliva onto the assay, what to do after the saliva is loaded, the time duration for the assay, and how to interpret the results.

Assays according to the invention have the capability of detecting biomarkers indicative of the COVID-19 disease at a very early stage of the SARS-CoV-2 virus infection process. Such early stage detection means or refers to any time from about one hour to about 14 days after onset of the earliest observed SARS-CoV-2 disease symptom(s). Symptoms and indicia of infection may include nonproductive cough, fever, dyspnea, myalgia, fatigue, and radiographic evidence of pneumonia.

In embodiments, assays according to the invention have the capability of detecting the biomarkers indicative of COVID-19 during the early stages of infection as well as early after the onset of COVID-19 symptoms (e.g., nonproductive cough, fever, dyspnea, myalgia, fatigue). Assays according to the invention have the capability of detecting the antibodies IgG1, IgG3, IgG4, Total IgG, IgM, IgA and the cytokines IL-2, IL-6, IL-10, IFN-γ, HGF, CSF-1, IL-18, and D-dimer starting within about one hour, 6 hours, 12 hours, or 1-2 weeks after onset of COVID-19 symptoms and continuing for several weeks with IgG1, IgG3, IgG4, Total IgG, IgM, IgA, IL-2, IL-6, IL-10, IFN-γ, HGF, CSF-1, IL-18, and D-dimer also capable of being detected at times from as little as 20 minutes following onset of COVID-19 symptoms to several weeks following manifestation of disease symptoms.

Still other types of assays may be implemented to detect biomarkers which correlate highly with the existence of a SARS-CoV-2 viral infection in a subject. Examples of assays include biosensor assays, multiplex assays, smart assays, microarray (e.g., lab-on-a-chip) assays to name a few. These different types of assays may be implemented to determine early SARS-CoV-2 virus infection, intermediate SARS-CoV-2 virus infection, and late SARS-CoV-2 virus infection. These assays may be useful to identify severity of the viral infection (i.e., early, intermediate, severe) by measuring changes over time in the concentration of the biomarkers IgG1, IgG3, IgG4, Total IgG, IgM, IgA, IL-2, IL-6, IL-10, IFN-γ, HGF, CSF-1, IL-18, and D-dimer.

A biosensor assay may be performed with a biosensor device. In a biosensor device, a saliva sample may be deposited by a technician in a reservoir plate. The saliva sample is delivered by means of a pump or gravity to a biochip connected to a processing device. The biochip may include sensors against which the saliva comes into contact. The sensors provide an electrical signal to the processing device which then provides a result which may include the biomarker type in the saliva sample as well as the biomarker concentration.

In other embodiments, a biosensor assay may be implemented with a smartphone optical biosensor. A smartphone optical biosensor represents a type of multichannel smartphone spectrometer (MSS) which can simultaneously assay multiple different saliva samples. A custom smartphone multi-view App may be implemented to control the optical sensing parameters and to align each saliva sample to the corresponding channel. The CCD or other optical device associated with the smartphone is capable of capturing images of the saliva samples and converting the transmission spectra in the visible wavelength range from 400 nm to 700 nm with the high resolution of 0.2521 nm per pixel. The CCD or other optical device provides a type of sensor which delivers data to the biosensor device. The performance of this MSS is capable of detecting and measuring the type and concentrations of biomarkers in the saliva sample. A multichannel smartphone optical biosensor is useful in high-throughput, point-of-care diagnostics with its minimal size, light weight, low cost, and data capture capabilities.

A lateral flow multiplexed assay strip capable of use in a multiplex assay may include a porous matrix enabling capillary flow of a saliva sample along the matrix including a sample pad, conjugate pad, and absorption pad as previously described. The conjugate pad may include two or more different types of labeled detection antibodies specific for a different target biomarker and may further include capture antibodies specific for each biomarker immobilized at respective test lines. If present in the saliva sample, each biomarker including a labeled detection antibody forms a complex with the capture antibody at the respective test line. Each unique label provides a different spectral emission indicative of the presence of the biomarker in the saliva sample which may be viewed with the naked eye or with some type of reader device.

A microarray assay is a type of multiplex lab-on-a-chip assay. Microarrays comprise a two-dimensional array of sensors on a solid substrate. The substrate is typically a glass slide or silicon thin-film cell. The array is capable of assaying large amounts of biological material, such as saliva, using high-throughput screening by means of miniaturized, multiplexed, and parallel processing and detection methods. The data from the sensors of the micro array is delivered to a processing device for output of information identifying the type and concentration of biomarker present in the saliva sample.

In embodiments, a method of treating a patient afflicted with COVID-19 may be implemented. For example, administration of an antiviral drug or inhibitor could lessen the symptoms of the COVID-19 disease in a subject and be useful in prevention, treatment, and management of a SARS-CoV-2 viral infection or other viral infection. Antiviral drugs or inhibitors may be directed against metabolic pathways responsible for excessive production of one or more of the biomarkers IgG1, IgG3, IgG4, Total IgG, IgM, IgA, IL-2, IL-6, IL-10, IFN-γ, HGF, CSF-1, IL-18, and D-dimer and the resultant autoimmune reaction which produces adverse symptoms in the patient. The concentration of the aforementioned biomarkers can correlate with efficacy of the drug or inhibitor in attenuating the pathway responsible for the biomarker production.

For example, a therapeutically effective dosage of a pharmaceutical agent, such as Sarilumab, Siltuximab, Tocilizumab, etc. to treat or improve the underlying conditions responsible for production of IgG1, IgG3, IgG4, Total IgG, IgM, IgA, IL-2, IL-6, IL-10, IFN-γ, HGF, CSF-1, IL-18, and D-dimer in the subject can be evaluated for efficacy by means of the invention. Reductions in the concentration of one or more of the biomarkers responsive to administration of the pharmaceutical agent can be interpreted as mitigating or ameliorating the disease.

In embodiments, an antiviral drug or inhibitor such as those above may be administered to a patient afflicted with COVID-19 or another viral infection. The period of time (e.g., hours, days, weeks, etc.) over which the administration occurs may be thought of as a treatment period. A saliva sample may be taken from the patient before administration of the drug or inhibitor to provide a baseline level of one or more of the biomarkers IgG1, IgG3, IgG4, Total IgG, IgM, IgA, IL-2, IL-6, IL-10, IFN-7, HGF, CSF-1, IL-18, and D-dimer. This initial saliva sample may be considered taken at a first time point. The biomarker in the initial saliva sample may be measured to determine the biomarker(s) present and their amount or concentration in the saliva.

At a subsequent or second time point, a further saliva sample (i.e., a second sample) may be taken from the patient. The biomarker in this further saliva sample may be measured to determine the biomarker(s) present and their amount or concentration in the saliva.

Next, the measured amounts of the one or more of IgG1, IgG3, IgG4, Total IgG, IgM, IgA, IL-2, IL-6, IL-10, IFN-γ, HGF, CSF-1, IL-18, and D-dimer biomarker taken at the first and second time points are compared for changes. In other words and in this example, the biomarker concentrations “before” administration of the drug or inhibitor and “after” the administration are compared and changes are memorialized. Changes in the biomarkers which correlate with lessening of the symptoms of COVID-19 in the patient are indicative that the patient is responsive to treatment with the antiviral drug or inhibitor.

Those skilled in the art will realize that it is occasionally necessary to make routine variations to the dosage of the pharmaceutical agent depending on age, route of administration, and condition of the patient such as age, weight, and clinical condition of the recipient patient. Methods, systems, and kits according to the invention are capable of providing information necessary to this adjustment process by providing opportunities to determine the severity and progression of COVID-19 in a subject.

Without wishing to be bound by any particular theory, it is believed that certain factors associated with the salivary biomarkers and biomarker combinations disclosed herein uniquely contribute to the strong correlation of the biomarkers with the existence of the COVID-19 disease in a subject. Isolation and identification of such biomarkers in saliva by means of biomarker-specific agents and antibodies permits implementation of assaying methods, systems, and kits capable of providing detection of SARS-CoV-2 virus and for the diagnosis, prognosis, monitoring, treatment, and management of COVID-19 disease in a subject with a high degree of certainty.

Antibody and cytokine biomarkers as described herein are by-products of the immune reaction to SARS-CoV-2 and the present inventors have discovered that they can be used to detect COVID-19 in asymptomatic and symptomatic subjects. Sarbecoviruses (e.g., SARS-CoV-2) express a large glycoprotein known as spike protein (S, a homotrimer), which mediates binding to host cells via interactions with the human receptor angiotensin converting enzyme 2 (ACE2). The spike protein is highly immunogenic with the receptor-binding domain (RBD) being the target of many neutralizing antibodies. Angkana T. Huang, et al., A Systematic Review of Antibody Mediated Immunity to Coronaviruses: Antibody Kinetics, Correlates of Protection, and Association of Antibody Responses with Severity of Disease, medRxiv.org (2020).

Individuals infected with coronaviruses typically produce neutralizing antibodies to the viral particle such as IgG1, IgG3, IgG4, Total IgG, IgM, and IgA and a neutralizing response has been demonstrated for SARS-CoV-2 in the cases of individual subjects from within a few hours after infection with SARS-CoV-2 to about 20 days thereafter. The present inventors have theorized that IgG3 might bind with both nucleocapsid protein (N-protein) and spike protein (S-protein) making the IgG3 biomarker particularly powerful in COVID-19 detection. Further, it is theorized that IgM is a strong indicator of COVID-19 because that antibody is existent shortly after infection, peaking within two weeks thereafter. It is thought by the present inventors that IgM binds with both the E protein and N protein of the viral particle. For infection by human coronaviruses, these and the other described antibody byproducts of the adaptive immune response have been linked to protection against infection for a period of time and, according to the present invention, their detection can be utilized to identify COVID-19.

Also responsive to a SARS-CoV-2 infection, are increased circulating levels of pro-inflammatory cytokines and chemokines such as IL-2, IL-6, IL-10, IFN-γ, HGF, CSF-1, IL-18, and D-dimer as well as other inflammatory signatures. In a normal immune reaction, the primary purpose of these biomarkers is to suppress inflammation. In SARS patients, however, these biomarkers are associated with pulmonary inflammation and lung involvement.

It is thought by the present inventors that IL-6 might induce the hyper-innate inflammatory response due to the SARS-CoV-1 invasion of the respiratory tract and that the same effect could occur responsive to SARS-CoV-2 responsible for COVID-19. In SARS-CoV-1 of the structural proteins, namely, nucleocapsid N, spike S, envelope E, and membrane M, only the nucleocapsid protein (N) significantly induced the activation of IL-6 promoter which plays a protective and essential role in the resolution process. Those same viral proteins exist with SARS-CoV-2, suggesting to the present inventors that the presence of IL-6 can be powerfully indicative of COVID-19 in a subject.

Accordingly, the present inventors have recognized that the biomarkers and biomarker combinations described herein can be utilized as indicative of COVID-19 in a subject. Detection of the combination of IgG3, IgM and IL6 is a particularly powerful indicator COVID-19 in the subject.

The present inventors have discovered that the biomarkers and biomarker combinations indicative of COVID-19 are optimally detected in saliva thereby representing a departure from current blood-based testing. Saliva is a bodily fluid which is more stable than blood serum and, unlike blood, saliva is inclusive of all cytokines and antibodies produced in response to SARS-CoV-2. Blood samples can yield inconsistent and less reproducible types and amounts of immune reaction biomarkers resulting in false negatives in serum-based COVID-19 assays. In contrast, saliva samples produce specimens with repeatable types and amounts of immune reaction biomarkers, avoiding the false negatives associated with blood and thereby providing a more accurate determinant of COVID-19 in a subject.

Methods, systems, and kits according to the invention may have some or all of the following advantages:

Broad applicability/high diagnostic value: Methods, systems, and kits according to the invention have a high sensitivity for, and are capable of identifying, a broad range of biomarkers indicative of COVID-19 in saliva samples. Biomarker examples are IgG1, IgG3, IgG4, Total IgG, IgM, IgA, IL-2, IL-6, IL-10, IFN-γ, HGF, CSF-1, IL-18, and D-dimer, separately and in combination.

Availability of components/low cost: In addition, antibodies capable of binding to, and detecting, the target salivary biomarker antigens IgG1, IgG3, IgG4, Total IgG, IgM, IgA, IL-2, IL-6, IL-10, IFN-γ, HGF, CSF-1, IL-18, and D-dimer are readily available, providing opportunities for a lower cost assay. Specifically, high-purity monoclonal or polyclonal antibodies to these biomarkers can be produced easily from mouse or rabbit sources. These detection antibodies are capable of being coated with colloidal gold and other labels for ease of identification in many different assays according to the invention.

Rapid results/ease of use: Methods, systems, and kits according to the invention are capable of detecting the target salivary biomarkers and providing results potentially within about 2 to about 30 minutes after starting the assay. In embodiments based on LFA assays, the user merely places saliva on the sample pad and waits for the result.

Methods, systems, and kits according to the invention can be portable and utilized and implemented wherever needed. Laboratory equipment and technical training are unnecessary. Methods, systems, and kits according to the invention, are suitable for clinical and home use, can quickly screen patients, and are suitable for on-site general screening and epidemiological investigation.

High accuracy: Methods, systems, and kits according to the invention yield reproducible and accurate results using many different types of assays such as LFA and ELISA assays.

Good stability: The saliva used with methods, systems, and kits according to the invention is stable. The saliva can be stored at −10° C. to 50° C. for months providing an opportunity to conduct multiple assays on the same sample over time which can be useful to verify the severity and progress of COVID-19 in a subject.

Noninvasiveness: Saliva is easily harvested from a subject and saliva collection is far less invasive to a subject than is collection of blood or another fluid or substance.

It can be appreciated that methods, systems, and kits according to the invention provide opportunities for improvements in healthcare for individual subjects, and groups of subjects, with respect to detection, diagnosis, monitoring, and treatment of the COVID-19 disease and potentially other viral infections.

### EXAMPLES

The following studies and the examples and data are provided to illustrate the invention, but are not intended to limit the scope of the invention in any way.

### Example 1—Efficacy of Biomarkers in Detection of COVID-19

Example 1 was conducted to evaluate and identify certain biomarkers and biomarker combinations in a subject indicative that the subject is afflicted with COVID-19. A diagnosis may be made based on this information. Specifically, Example 1 sought to determine the concentrations of IgG1, IgG3, IgG4, Total IgG, IgM, IgA, IL-2, IL-6, IL-10, IFN-γ, HGF, CSF-1, IL-18, and D-dimer in saliva existent in groups of healthy (i.e., control) subjects and groups of subjects who had been confirmed as testing positive for COVID-19. Differences in the concentration of the aforementioned biomarkers between healthy and COVID-19 subjects are indicative of affliction with the disease.

As indicated in Table 1, thirty (30) healthy subjects and fifty-seven (57) subjects diagnosed with COVID-19 were studied in Example 1. RT-PCR was used to confirm that each of the 57 subjects was positive for SARS-CoV-2. In the example, viral RNA was taken from clinical samples by automatic extractor. MagCore HF16 (RBC Bioscience, Taiwan) and Hamilton Microlab Starlet (Hamilton Company, Bonaduz, Switzerland). RNA amplification was made using an Allplex 2019-nCoV assay real-time PCR platform (Seoul, South Korea). The RT-PCR was performed in accordance with the manufacturer's instructions for performance of the assays and interpretation of the results.

Saliva samples were collected from each subject, including the subjects in COVID-19 and in control groups. Ten to fifteen minutes prior to collection of unstimulated saliva samples, subjects were asked to rinse orally with water. At the time of sample collection, each subject was asked to relax for 5-15 minutes. They were then seated in a bent forward position in an ordinary chair and asked to put their tongues on the lingual surfaces of the upper incisors and to allow the saliva to drip into sterile plastic (glass) tubes treated with 50 g of 2% sodium azide solution, to prevent microbial decomposition of saliva. The tubes were held to the lower lip for 10 minutes resulting in a collection of 1-5 ml of saliva per individual. Saliva samples were then centrifuged using a Sorvall RT6000D centrifuge (Sorvall, Minn.) at 1800 rpm for 5 minutes to remove debris and were then immediately frozen at −80° C., awaiting further analysis.

The saliva samples taken from members of the group of healthy subjects and from members of the group known to be afflicted with COVID-19 were assayed for the presence of the biomarkers by means of enzyme-linked immunosorbent assays (ELISA) specific for each biomarker.

Concentrations of IgG1, IgG3, IgG4, Total IgG, IgM, and IgA were measured by ELISA kits from Thermofisher Scientific. Thermofisher Scientific assay kits EHIGG1, BMS2094, BMS2095, BMS2091, 88-50620-22, and BMS2096 respectively.

IL-6 levels in each saliva sample were measured using a commercial ELISA kit from Thermofisher Scientific, (IL-6 ELISA kit, High sensitivity, cat. No. BMS213HS). Levels of IL-2 were measured using an ELISA kit from Sigma Immunochemicals. The levels of IL-10 were measured by using ELISA kits from Diaclone SAS. The levels of D-dimer were estimated by using a D-dimer Alpha ELISA kit from Perkin Elmer (Product-No: AL290 C/F). Salivary levels of IFN-γ and CSF-1 were measured by using ELISA kits from R&D Systems. HGF and IL-18 levels in the saliva were determined using ELISA kits from R&D Systems (Quantikine@ human HGF immunoassay and IL-18 ELISA kit, cat. No. KHC018).

As described in the analysis below, “area under the curve” (AUC) using a receiver operating characteristic analysis was also implemented to determine the screening ability of the salivary biomarkers to predict COVID-19. Stated differently, the area under the receiver operating characteristic curve (AUC) was calculated for determining the prognostic accuracy of the salivary biomarkers. Data were analyzed by using Statistical Package for the Social Sciences (SPSS version 22; IBM Corporation, Armonk, N.Y.)

Table 1 provides demographics and baseline characteristics of the healthy control subjects and the subjects afflicted with COVID-19. As is shown in Table 1, the average age of the healthy subjects was 34.7 years (standard deviation SD 5.3) while the average age of the COVID-19 subjects was 36.8 years of age (standard deviation SD 3.9). The relatively “young” age of the subjects is relevant because people of this age group tend to have more robust immune systems which might suppress outward manifestations of COVID-19 rendering the person “asymptomatic”. These data indicate that the biomarkers would be expected to have utility in identifying COVID-19 disease in younger people who are asymptomatic. These asymptomatic people may be so-called “super spreaders”, that is people who do not believe they have the disease and yet inadvertently spread the disease to others.

Table 1 further indicates that the subjects of the COVID-19 group had contacts with 26 other people suspected of having COVID-19 and that an average of 14 days had elapsed between that contact and collection of the saliva samples. Therefore, the COVID-19 would have been well-advanced in each member of the COVID-19 group. Table 1 indicates that members of the COVID-19 group had symptoms indicative of the disease.

TABLE 1
Demographics and Baseline Characteristics of the Subjects

| Characteristics | Healthy Subjects | COVID-19 Subjects |
|---|---|---|
| Age (years) (SD) | 34.7 (5.3) | 36.8 (3.9) |
| Gender (M/F) | 16/14 | 30/27 |
| Exposure (close contacts) | N/A | 26 |
| Days after exposure | N/A | 14 |
| Subjects with systemic | N/A | 8 |
| diseases | | |

Signs and Symptoms

| | | |
|---|---|---|
| Fever | None | 30 |
| Fatigue | None | 10 |
| Dry cough | None | 26 |
| Anorexia | None | 9 |
| Headache | None | 18 |
| Diarrhea | None | 12 |
| Myalgia | None | 10 |
| Chest stuffiness | None | 8 |

TABLE 2A
Salivary Biomarkers in Healthy Subjects and COVID-19 Subjects

| | IgG1 | IgG3 | IgG4 | Total IgG | IgM | IgA | IL-2 |
|---|---|---|---|---|---|---|---|
| | mg/dl) | (ng/ml) | (mg/l) | (mg/dl) | (mg/dl) | (mg/dl) | (pg/ml) |
| Healthy | | | | | | | |
| Subjects | | | | | | | |
| Mean | 10.2 | 126.4 | 14.8 | 24.5 | 123 | 220 | 7.5 |
| (SD) | (1.3) | (32.1) | (1.3) | (5.2) | (21) | (30.3) | (1.4) |
| Range | 4.2-40.2 | 10-1460 | 10.8-20.3 | 10.4-65.3 | 120-231 | 140-269 | 5.6-11.9 |
| COVID-19 | | | | | | | |
| Subjects | | | | | | | |
| Mean | 89.6 | 453.8 | 24.7 | 72.5 | 252.4 | 304.5 | 32.6 |
| (SD) | (6.9) | (45.5) | (3.2) | (11.4) | 26.4) | (24.6) | (5.4) |
| Range | 39.6-100.6 | 136.5-2178 | 23-108 | 66-265 | 250-560 | 300-870 | 27-168 |

TABLE 2B
Salivary Biomarkers in Healthy Subjects and COV1D-19 Subjects

| | IL-6 | IL-10 | IFN-γ | HGF | CSF-1 | IL-18 | D-dimer |
|---|---|---|---|---|---|---|---|
| | (pg/ml) | (pg/ml) | (ng/ml) | (ng/ml) | (pg/ml) | (pg/ml) | (ng/ml) |
| Healthy | | | | | | | |
| Subjects | | | | | | | |
| Mean | 2.5 | 2.7 | 45.3 | 0.89 | 1267.3 | 256.8 | 145.6 |
| (SD) | (0.8) | (1.2) | (12.3) | (0.27) | (242.5) | (45.3) | (13.5) |
| Range | 1.2-40 | 0.9-10 | 34.2-134.6 | 0.64-1.87 | 234.42-1862.08 | 134-456 | 35-452 |
| COVID-19 | | | | | | | |
| Subjects | | | | | | | |
| Mean | 46.7 | 12.6 | 212.4 | 2.78 | 1345.8 | 375.5 | 509.3 |
| (SD) | (3.6) | (2.7) | (15.6) | (0.42) | (109.5) | (36.8) | (46.4) |
| Range | 12-59 | 11-68 | 200-563 | 1.9-4.8 | 1200-4532 | 300-1489 | 450-2543 |

Tables 2A and 2B provide mean amounts of the biomarkers, the standard deviation (SD), and the range of the amounts of the biomarkers. Units of measure are provided. As can be appreciated, there is a significant difference in the biomarker concentrations in the subjects of the healthy and COVID-19 groups. Tables 2A and 2B demonstrate that the concentration of the separate biomarkers IgG1, IgG3, IgG4, Total IgG, IgM, IgA, IL-2, IL-6, IL-10, IFN-γ, and D-dimer were greater in members of the COVID-19 group of subjects than in the healthy, control group of subjects. For example, the mean value of IL-6 in healthy subjects is 2.5 pg/ml while the IL-6 in COVID-19 subjects is 46.7 pg/ml. The biomarker concentrations of the healthy subjects provide a reference value or amount or level of biomarker against which subjects afflicted with COVID-19 can be compared.

Tables 2A and 2B demonstrate that salivary IgG1, IgG3, IgG4, Total IgG4, IgM, IgA, IL-2, IL-6, IL-10, IFN-γ, CSF-1, HGF and IL 18, D-dimer concentrations could differentiate healthy control subjects from the COVID-19 subjects, with an AUC (0.85-0.99, p=0.005). The separate six biomarkers consisting of IgG3, IgM, IL-6, CSF-1, HGF and D-dimer had particularly high diagnostic values in differentiating healthy control subjects from the COVID-19 subjects with an AUC value respectively of 0.98, 0.89, 0.96, 0.92, 0.96 and 0.91, p=0.005. These data indicate that the aforementioned separate biomarkers can be implemented as indicative of COVID-19 for use in early detection of COVID-19 for the diagnosis, prognosis, monitoring, treatment, and management of the SARS-CoV-2 virus and/or other types of viral infections.

Table 3 below demonstrates that the specificity and sensitivity of the saliva-based biomarkers IgG3, IL-6, and IgM, are highly predictive of the existence of COVID-19 disease in a subject. In Table 3, PPV refers to positive predictive value and NPV refers to negative predictive value.

TABLE 3
Predictive Value of Salivary IL-6, IgM, and IgG3 in
Detection of COVID-19

| Biomarker | Cutoff | Sensitivity | Specificity | PPV | NPV |
|---|---|---|---|---|---|
| IgG3 | 130 ng/ml | 0.86 | 0.85 | 0.85 | 0.84 |
| IL-6 | 40 pg/ml | 0.84 | 0.83 | 0.82 | 0.83 |
| IgM | 123 mg/dl | 0.82 | 0.80 | 0.81 | 0.80 |

The data of Table 3 demonstrate that IgG3, IL-6, and IgM are each separately highly sensitive and specific for detection of COVID-19 in a subject. The cutoff level values indicated in Table 3 represent useful biomarker concentrations which may be used to characterize the subject as having or not having COVID-19. The cutoff level value may represent a reference value or amount or level of biomarker derived from subjects not afflicted with COVID-19. Biomarker concentrations below the cutoff level may be indicative that the subject does not have COVID-19 and biomarker concentrations above the cutoff level may be indicative that the subject is infected with the SARS-CoV-2 virus and is positive for COVID-19.

It is expected that a useful cutoff level may also be within a range. For example, a cutoff value for IgG3 may in the range of about 120 ng/ml to about 200 ng/ml, with a more preferred range being about 130 ng/ml to about 180 ng/ml. A useful cutoff level value range for IL-6 may be about 15 pg/ml to about 60 pg/ml, with a more preferred range being about 20 pg/ml to about 40 pg/mIl A useful cutoff level value range for IgM may be about 120 mg/dl to about 140 mg/dl, with a range of 123 mg/dl to about 130 mg/dl being more desirable.

Table 4 which follows is directed to efficacy of examples of pairs of biomarkers listed in Tables 2A and 2B in the early detection of COVID-19 and the existence of the SARS-CoV-2 virus in a human subject. The exemplary combinations and calculated AUC values are in Table 4.

TABLE 4
Salivary Biomarker Combinations Useful in Detection of COVID-19

| | Biomarker Combination | AUC |
|---|---|---|
| 1 | IgG3 and IL-6 | 0.99 |
| 2 | IgG3 and IgM | 0.98 |
| 3 | IgM and IL-6 | 0.98 |
| 4 | IgG3 and CSF-I | 0.98 |
| 5 | IgG3 and HGF | 0.98 |
| 6 | IgG3 and D-dimer | 0.97 |
| 7 | IL-6 and CSF-1 | 0.96 |
| 8 | CSF-I and HGF | 0.95 |
| 9 | HGF and D-diiner | 0.95 |

Table 4 provides calculated AUC values based on Tables 2A and 2B and demonstrates that biomarker combinations are useful in differentiating healthy control subjects from the COVID-19 subjects with an AUC value as reported in Table 3 and a p value of 0.0050 (p=0.0050). Table 4 demonstrates that the examples of paired biomarkers would be highly effective at detecting SARS-CoV-2 virus in a human subject.

### Example 2—Efficacy of Biomarkers in Detection of Severity of COVID-19

Example 2 was conducted to evaluate the capability of salivary biomarkers to detect the existence of COVID-19 in asymptomatic subjects and to differentiate between degrees of severity of COVID-19 in subjects with mild, intermediate, and severe levels of a SARS-CoV-2 infection.

As indicated in Table 5, saliva samples were taken from 100 subjects, including 30 healthy (i.e., control) subjects, 20 asymptomatic subjects infected with SARS-CoV-2, 36 subjects with mild symptoms of COVID-19, and 14 subjects with severe COVID-19 symptoms. Asymptomatic subjects and COVID-19 subjects were confirmed by RT-PCR assay from nasal and pharyngeal swab samples. Saliva samples were collected from the subjects in the same manner as described in Example 1.

Table 5 provides demographics and baseline characteristics of the healthy control subjects and the subjects afflicted with COVID-19. As is shown in Table 4, the average age of the healthy subjects was 36.8 years, the age of the asymptomatic COVID-19 subjects was 35.8 years, the age of the mild COVID-19 subjects was 36.4 years, and the age of the severe COVID-19 subjects was 37.3 years. The mid-30s average age of the subjects is relevant for the same reasons as described in connection with Example 1 and is particularly important because biomarkers as described herein are useful in identifying healthy appearing asymptomatic people afflicted with COVID-19 who could spread the disease to others.

Table 5 further indicates that the subjects of the COVID-19 group had contacts with other people suspected of having COVID-19 and that at least 11 days had elapsed between that contact and collection of the saliva samples. Therefore, the COVID-19 would have been well-advanced in each member of the COVID-19-afflicted group. Like Table 1, Table 5 indicates that members of the COVID-19 group had symptoms indicative of the disease.

TABLE 5
Demographics and Baseline Characteristics of the Subjects

| | | Asymptomatic | Mild | Severe |
|---|---|---|---|---|
| | Healthy | COVID-19 | COVID-19 | COVID-19 |
| | Subjects | Subjects | Subjects | Subjects |
| Characteristics | n = 50 | n = 20 | n = 36 | n = 14 |
| Age (years) (SD) | 36.8(4.2) | 35.8(5.1) | 36.4(4.3) | 37.3(5.1) |
| Gender (M/F) | 25/25 | 10/10 | 17/19 | 8/6 |
| Exposure (close | N/A | 14 | 30 | 11 |
| contacts) in number | | | | |
| Days after exposure | N/A | 12 | 14 | 16 |
| Subjects with | N/A | 6 | 9 | 12 |
| systemic diseases | | | | |

Signs and symptoms

| | | | | |
|---|---|---|---|---|
| Fever | None | None | 32 | 13 |
| Fatigue | None | None | 2 | 2 |
| Dry cough | None | None | 25 | 9 |
| Anorexia | None | None | 9 | 1 |
| Headache | None | None | 21 | 9 |
| Diarrhea | None | None | 4 | 3 |
| Myalgia | None | None | 5 | 4 |
| Chest stuffiness | None | None | 3 | 12 |

TABLE 6A
Salivary Biomarkers in Healthy Subjects and COV1D-19 Subjects

| | IgG1 | IgG3 | IgG4 | Total | IgM | IgA | IL-2 |
|---|---|---|---|---|---|---|---|
| | (mg/ | (ng/ | mg/ | IgG | (mg/ | (mg/ | (pg/ |
| | dl) | ml) | dl) | (mg/l) | dl) | dl) | ml) |
| Healthy | | | | | | | |
| Subjects | | | | | | | |
| Mean | 11.4 | 132.5 | 5.2 | 26.5 | 125.4 | 220.4 | 7.4 |
| (SD) | (3.2) | (31.6) | (1.4) | (3.3) | (15/7) | (31.6) | (1.6) |
| Asymp- | | | | | | | |
| tomatic | | | | | | | |
| Subjects | | | | | | | |
| Mean | 18.9 | 245.6 | 15.3 | 33.5 | 200.5 | 278.6 | 12.5 |
| (SD) | (4.3) | (25.4) | (3.6) | (4.2) | (16/6) | (20.4) | (3.4) |
| Mild | | | | | | | |
| Subjects | | | | | | | |
| Mean | 37.6 | 407.5 | 16.7 | 36.7 | 231.7 | 308.4 | 14.6 |
| (SD) | (2.8) | (21.4) | (3.6) | (5.1) | (23.5) | (17.5) | (4.4) |
| Severe | | | | | | | |
| Subjects | | | | | | | |
| Mean | 49.5 | 489.4 | 20.5 | 65.6 | 278.5 | 326.3 | 37.8 |
| (SD) | (10.5) | (34.5) | (2.6) | (10.4) | (26.7) | (25.7) | (5.7) |

TABLE 6B
Salivary Biomarkers in Healthy Subjects and COVID-19 Subjects

| | | | | | | | D- |
|---|---|---|---|---|---|---|---|
| | IL-6 | IL-10 | IFN-γ | HGF | CSF-1 | IL-18 | dimer |
| | (pg/ | (pg/ | (ng/ | (ng/ | (pg/ | (pg/ | (ng/ |
| | ml) | ml) | ml) | ml) | ml) | ml) | ml) |
| Healthy | | | | | | | |
| Subjects | | | | | | | |
| Mean | 2.6 | 2.6 | 47.4 | 0.98 | 1287.7 | 252.4 | 153.3 |
| (SD) | (1.2) | (1.6) | (11.6) | (0.23) | (272.5) | (13.6) | (41.3) |
| Asymp- | | | | | | | |
| tomatic | | | | | | | |
| Subjects | | | | | | | |
| Mean | 46.4 | 10.8 | 108.5 | 1.76 | 1598.4 | 304.5 | 234.7 |
| (SD) | (3.7) | (1.7) | (13.5) | (0.42) | (234.1) | (12.7) | (36.8) |
| Mild | | | | | | | |
| Subjects | | | | | | | |
| Mean | 53.1 | 13.4 | 110.7 | 2.01 | 2367.3 | 327.4 | 456.4 |
| (SD) | (2.8) | (2.2) | (14.6) | (0.42) | (209.3) | (31.7) | (42.5) |
| Severe | | | | | | | |
| Subjects | | | | | | | |
| Mean | 63.8 | 18.6 | 245.8 | 3.51 | 3345.4 | 386.7 | 543.6 |
| (SD) | (4.7) | (3.2) | (30.6) | (0.64) | (214.3) | (35.4) | (48.9) |

Tables 6A and 6B provide mean amounts of the biomarkers and the standard deviation (SD) of the amounts of the biomarkers. Units of measure are provided. As can be appreciated, there is a significant difference in the biomarker concentrations in the subjects of the healthy as compared with the asymptomatic, mild, and severe COVID-19 groups. Tables 6A and 6B demonstrate that the concentration of the separate biomarkers IgG1, IgG3, IgG4, Total IgG, IgM, IgA, IL-2, IL-6, IL-10, IFN-γ, and D-dimer were greater in members of all COVID-19 group as compared with the healthy, control group of subjects. Tables 6A and 6B show that there are increased concentrations of the biomarkers in asymptomatic COVID-19 subjects as compared with the healthy subjects. The healthy subjects, therefore, provide a reference value or amount for each biomarker that can be used for comparison. Such reference values provide a basis to detect asymptomatic carriers, and potential spreaders, of the SARS-CoV-2 virus.

Tables 6A and 6B further indicate that salivary IgG1, IgG3, IgG4, Total IgG4, IgM, IgA, IL-2, IL-6, IL-10, IFN-7, CSF-1, HGF and IL 18, D-dimer concentrations increase as the severity of the COVID-19 disease increases and can differentiate asymptomatic subjects from subjects with either mild or severe COVID-19. Such biomarker concentrations can also differentiate subjects with mild COVID-19 from people with severe cases of the disease.

Area under the curve (AUC) using a receiver operating characteristic analysis was also used to determine the screening ability of the salivary biomarkers of Tables 6A and 6B to determine and predict the severity of the disease. That is, the area under the receiver operating characteristic curve (AUC) was calculated for determining the prognostic accuracy of the salivary biomarkers in determining the severity of the COVID-19 disease. The AUC for the biomarkers of Example 2 was determined to be 0.83-0.99, p=0.005.

Tables 7A and 7B which follow are based on the results of Tables 6A and 6B and show that no correlations were found between subject age and the listed biomarkers. Accordingly, the biomarkers of Tables 7A and 7B can be used effectively for detection of COVID-19 and for the diagnosis, prognosis, monitoring, treatment, and management of the SARS-CoV-2 virus and/or other types of viral infections.

TABLE 7A
Correlations Between Biomarkers and Age Among all Subjects

| | IgG1 | IgG3 | IgG4 | Total IgG | IgM | IgA | IL-2 |
|---|---|---|---|---|---|---|---|
| R | 0.05 | 0.06 | 0.07 | 0.04 | 0.01 | 0.02 | 0.03 |
| P value | 0.98 | 0.73 | 0.71 | 0.64 | 0.75 | 0.82 | 0.74 |

TABLE 7B
Correlations Between Biomarkers and Age Among all Subjects

| | IL-6 | IL-10 | IFN-γ | HGF | CSF-1 | IL-18 | D-dimer |
|---|---|---|---|---|---|---|---|
| R | 0.04 | 0.05 | 0.01 | 0.02 | 0.03 | 0.02 | 0.08 |
| P value | 0.69 | 0.61 | 0.78 | 0.92 | 0.84 | 0.86 | 0.87 |

### Example 3—Efficacy of Multiple Biomarker Combinations in Detection of COVID-19

Example 3 demonstrates that further combinations of biomarkers are efficacious with respect to detection of COVID-19 and for the diagnosis, prognosis, monitoring, treatment, and management of the SARS-CoV-2 virus and/or other types of viral infections. The combinations may be implemented as part of a biomarker panel on a solid support used, for example, in an ELISA type assay. According to Example 3, a statistical comparison of the two populations (by the combination of the salivary biomarkers in Examples 1 and 2) was performed using the two-tailed t-test using GraphPad Prism for Windows, v. 5.01. (GraphPad Software, San Diego, Calif.) Receiver operating characteristic curves (ROC) were generated using the R software environment for statistical computing and graphics. (R Foundation for Statistical Computing, Vienna, Austria)

Tables 8A and 8B which follow provide an ROC analysis and diagnostic performance analysis for various salivary biomarker combinations, namely, IgG3 (A), IL-6 (B), IgM (C), HGF (D), CSF-1(E), D-dimer (F), IL 18 (G), IgA (H), IgG1 (I), IgG4 (J), IL-2 (K), IL-10 (L), IFN-γ (M) and total IgG (N). The exemplary combinations may be implemented for the diagnosis of, and discrimination between, subjects with COVID-19 and healthy control subjects. Tables 8C and 8D provide cutoff values for the individual biomarkers making up each combination with units of measure also provided.

TABLE 8A
ROC Analysis of Salivary Biomarker
Combinations in Detection of COVID-19

| | A | AB | ABC | ABCD | ABCDE | ABCDEF | ABCDEFG |
|---|---|---|---|---|---|---|---|
| AUC | 0.96 | 0.97 | 0.98 | 0.99 | 0.99 | 0.99 | 0.99 |
| Sensi- | 0.86 | 0.86 | 0.88 | 0.92 | 0.95 | 0 .96 | 0.97 |
| tivity | | | | | | | |
| Speci- | 0.85 | 0.87 | 0.87 | 0.93 | 0.94 | 0.95 | 0.97 |
| ficity | | | | | | | |

TABLE 8B
ROC Analysis of Salivary Biomarker
Combinations in Detection of COVID-19

| | ABCD- | ABCD- | A to | A to | A to | A to | A to |
|---|---|---|---|---|---|---|---|
| | EFGH | EFGHI | J | K | L | M | N |
| AUC | 0.99 | 0.99 | 0.99 | 0.99 | 0.99 | 0.99 | 0.99 |
| Sensi- | 0.98 | 0.98 | 0.98 | 0.98 | 0.98 | 0.98 | 0.98 |
| tivity | | | | | | | |
| Speci- | 0.98 | 0.99 | 0.99 | 0.99 | 0.99 | 0.99 | 0.99 |
| ficity | | | | | | | |

TABLE 8C
Biomarker Cutoff Level Values

| | IgG1 | IgG3 | IgG4 | Total IgG | IgM | IgA | IL-2 |
|---|---|---|---|---|---|---|---|
| | (mg/dl) | (ng/ml) | (mg/dl) | (mg/l) | (mg/dl) | (mg/dl) | (pg/ml) |
| Cut- | 20 | 130 | 13 | 32 | 123 | 268 | 11 |
| off | | | | | | | |

TABLE 8D
Biomarker Cutoff Level Values

| | IL-6 | IL-10 | IFN-γ | HGF | CSF-1 | IL-18 | D-dimer |
|---|---|---|---|---|---|---|---|
| | (pg/ml) | (pg/ml) | (ng/ml) | (ng/ml) | (pg/ml) | (pg/ml) | (ng/ml) |
| Cut- | 40 | 9 | 70 | 1.3 | 1469 | 302 | 207 |
| off | | | | | | | |

The ROC analysis established diagnostic sensitivity and specificity for COVID-19 infections and other virus infections as shown in Tables 8A and 8B. The combination model including IgG3, IL-6, and IgM (combination ABC) demonstrates excellent diagnostic values for diagnosis of COVID-19 and other viral infections and is an especially efficacious model given that COVID-19 can be detected with a high level of confidence with just three biomarkers. Use of relatively fewer biomarkers (e.g., three biomarkers) is desirable for cost reduction and simplicity purposes.

Another excellent combination model according to Table 8A includes IgG3, IL-6, IgM, HGF, CSF-1, and D-dimer (combination A to F). This six biomarker combination also has high diagnostic values for diagnosis of COVID-19 and other viral infections. Based on the data of Tables 8A and 8B, it can be expected that the combination of any two or more of the biomarkers in Tables 8A and 8B would have high diagnostic values for detection of COVID-19 and for the diagnosis, prognosis, monitoring, treatment, and management of the SARS-CoV-2 virus in a subject.

Table 8E below provides a further ROC analysis and diagnostic performance analysis for further salivary biomarker combinations utilizing the biomarkers identified above in connection with Tables 8A and 8B. Table 8C demonstrates that IgG3 and IgA in paired combination and in combination with the additional biomarkers may be implemented for the diagnosis of, and discrimination between, subjects with COVID-19 and healthy control subjects.

TABLE 8E
ROC Analysis of Salivary Biomarker
Combinations in Detection of COVID-19

| | A | AH | AHC | AHCM | AHCFM | AHCFKM |
|---|---|---|---|---|---|---|
| AUC | 0.96 | 0.97 | 0.98 | 0.99 | 0.99 | 0.99 |
| Sensitivity | 0.86 | 0.86 | 0.87 | 0.90 | 0.91 | 0.95 |
| Specificity | 0.85 | 0.86 | 0.87 | 0.90 | 0.92 | 0.94 |

### Example 4—Reproducibility of Results

Example 4 was conducted to evaluate the reproducibility and stability of salivary biomarkers. Reproducibility of results is important, for example, to confirm that examples of salivary biomarkers can be used to reliably monitor the progression of COVID-19 in a subject over a period of time. A salivary biomarker sample with reproducible usage may provide a baseline by which to measure a subject's improvement or deterioration.

According to Example 4, saliva samples from twenty (20) healthy subjects and separately twenty (20) COVID-19 subjects were obtained. The samples were randomly arranged and labeled such that the laboratory could not identify the subjects sampled.

For each analysis, the assay reproducibility of blinded quality control replicates was examined using the coefficient of variation (CV), a commonly used statistical analysis technique to describe laboratory technical error, and a determination was made of the effect of delayed sample processing on analyte concentrations in frozen samples at −80° C. (at twenty-four hours, seven days and fourteen days after sampling, i.e., reproducibility with delayed processing). Reproducibility was assessed over a one-week and two-week period for salivary biomarkers, by taking samples at seven days and fourteen days. The CV was determined by estimating the SD (standard deviation) of the quality control values, divided by the mean of these values, multiplied by 100. Inter-observer and intra-observer variances were estimated from repeated sample measurements using a random effects model, with sample identification number as the random variable.

To assess reproducibility, the ICC (Intraclass Correlation Coefficient) values were calculated by dividing the intra-observer variance by the sum of the within- and inter-observer variances. Ninety-five percent (95%) confidence intervals (CI) were also calculated. The inter- and intra-observer CVs were determined by taking the square root of the inter- and intra-observer variance components from the random effects mixed model on the ln [log] transformed scale, with approximate estimates derived by the eta method. (Rosner B, Fundamentals of Biostatistics, Duxbury (2006)) An ICC of <0.40 indicates poor reproducibility, an ICC of 0.40 to 0.8 indicates fair to good reproducibility, and an ICC of more than 0.8 indicates excellent reproducibility. Results are shown in Tables 9 and 10.

Table 9 provides ICCs calculated for delayed analysis and processing of a single frozen sample at day one, day seven, and day fourteen for salivary biomarkers in subjects. Table 10 provides ICCs calculated of samples tested at various time points (day one, day seven and day fourteen) in all subjects.

TABLE 9
Intraclass Correlation Coefficient - Single Saliva Sample in Subjects

| | Number of | Intra-observer | | | Inter-observer | | | ICC | | |
|---|---|---|---|---|---|---|---|---|---|---|
| | participants/ | CV (%) | | | CV (%) | | | (95% CIs) | | |
| Bio- | number of | Day | Day | Day | Day | Day | Day | Day | Day | Day |
| marker | time points | 1 | 7 | 14 | 1 | 7 | 14 | 1 | 7 | 14 |
| IgG1 | 40/3 | 1.3 | 1.6 | 1.7 | 2.2 | 2.3 | 2.5 | 0.93 | 0.92 | 0.91 |
| IgG3 | 40/3 | 1.4 | 1.4 | 1.5 | 2.4 | 2.6 | 2.8 | 0.92 | 0.90 | 0.90 |
| IgG4 | 40/3 | 1.6 | 1.7 | 1.7 | 2.1 | 2.2 | 2.6 | 0.94 | 0.93 | 0.92 |
| Total IgG | 40/3 | 1.2 | 1.4 | 1.5 | 2.3 | 2.4 | 2.6 | 0.93 | 0.92 | 0.90 |
| IgM | 40/3 | 1.3 | 1.3 | 1.6 | 2.5 | 2.6 | 2.7 | 0.92 | 0.91 | 0.90 |
| IgA | 40/3 | 1.5 | 1.4 | 1.5 | 2.4 | 2.7 | 2.8 | 0.94 | 0.93 | 0.92 |
| IL-2 | 40/3 | 1.3 | 1.4 | 1.5 | 2.6 | 2.8 | 3.1 | 0.92 | 0.91 | 0.91 |
| IL-6 | 40/3 | 1.2 | 1.7 | 1.8 | 2.4 | 2.5 | 2.9 | 0.93 | 0.92 | 0.91 |
| IL-10 | 40/3 | 1.4 | 1.4 | 1.5 | 2.5 | 2.6 | 2.8 | 0.90 | 0.91 | 0.90 |
| IFN-γ | 40/3 | 1.5 | 1/8 | 1.9 | 2.7 | 7.8 | 2.9 | 0.92 | 0.91 | 0.89 |
| CSF-1 | 40/3 | 1.3 | 1.5 | 1.6 | 2.3 | 2.5 | 2.6 | 0.91 | 0.90 | 0.89 |
| IL-18 | 40/3 | 1.3 | 1.4 | 1.7 | 2.9 | 2.9 | 3.1 | 0.89 | 0.87 | 0.86 |
| HGF | 40/3 | 1.6 | 1.7 | 1.9 | 2.7 | 2.8 | 2.9 | 0.94 | 0.93 | 0.91 |
| D-dimer | 40/3 | 1.5 | 1.8 | 1.9 | 2.6 | 2.7 | 2.8 | 0.92 | 0.91 | 0.90 |

TABLE 10
Intraclass Correlation Coefficient - Time Point Testing in All Subjects

| | Number of | Intra-observer CV | | | Inter-observer CV | | | ICC | | |
|---|---|---|---|---|---|---|---|---|---|---|
| | participants/ | (%) | | | (%) | | | (95% CIs) | | |
| | number of | Day | Day | Day | Day | Day | Day | Day | Day | Day |
| Biomarker | time points | 1 | 7 | 14 | 1 | 7 | 14 | 1 | 7 | 14 |
| IgG1 | 40/3 | 1.3 | 1.4 | 1.5 | 2.5 | 2.6 | 3.1 | 0.93 | 0.93 | 0.91 |
| IgG3 | 40/3 | 1.2 | 1.4 | 1.6 | 2.3 | 2.7 | 3.4 | 0.92 | 0.92 | 0.92 |
| IgG4 | 40/3 | 1.4 | 1.5 | 1.8 | 2.4 | 2.8 | 3.2 | 0.94 | 0.93 | 0.92 |
| Total IgG | 40/3 | 1.6 | 1.7 | 1.5 | 2.2 | 3.1 | 3.3 | 0.91 | 0.90 | 0.89 |
| IgM | 40/3 | 1.3 | 1.4 | 1.6 | 2.5 | 2.8 | 3.4 | 0.92 | 0.91 | 0.90 |
| IgA | 40/3 | 1.5 | 1.8 | 1.9 | 2.4 | 2.9 | 3.1 | 0.94 | 0.92 | 0.90 |
| IL-2 | 40/3 | 1.4 | 1.6 | 1.5 | 2.6 | 3.2 | 2.7 | 0.93 | 0.93 | 0.92 |
| IL-6 | 40/3 | 1.6 | 1.8 | 1.9 | 2.5 | 2.8 | 2.8 | 0.92 | 0.90 | 0.89 |
| IL-10 | 40/3 | 1.8 | 1.6 | 2.1 | 2.8 | 2.9 | 3.2 | 0.91 | 0.90 | 0.90 |
| IFN-γ | 40/3 | 1.6 | 2.1 | 1.7 | 2.5 | 3.1 | 3.3 | 0.93 | 0.91 | 0.87 |
| CSF-1 | 40/3 | 1.3 | 1.6 | 1.8 | 2.1 | 2.8 | 2.9 | 0.92 | 0.91 | 0.89 |
| IL-18 | 40/3 | 1.7 | 1.8 | 1.9 | 2.2 | 2.7 | 2.8 | 0.90 | 0.89 | 0.88 |
| HGF | 40/3 | 1.5 | 1.7 | 1.6 | 2.4 | 2.6 | 2.7 | 0.93 | 0.91 | 0.90 |
| D-dimer | 40/3 | 1.4 | 1.5 | 1.8 | 2.6 | 2.5 | 2.6 | 0.92 | 0.90 | 0.89 |

The data of Example 4 demonstrate that the ICCs for the range of salivary biomarkers were high (ICCs of 0.9-0.95), indicating good to excellent reproducibility and stability. Example 4 demonstrates that the biomarkers of the study are stable and easy to reproduce.

Those skilled in the art will recognize that numerous modifications and changes may be made to the preferred embodiments without departing from the scope of the claimed invention. It will, of course, be understood that modifications of the invention, in its various aspects, will be apparent to those skilled in the art, some being apparent only after study, others being matters of routine mechanical, chemical, and electronic design. No single feature, function, or property of the preferred embodiments is essential. Other embodiments are possible, their specific designs depending upon the particular application. As such, the scope of the invention should not be limited by the particular embodiments herein described, but should be defined only by the appended claims and equivalents thereof.

1. A ready-to-use kit for detecting salivary biomarkers in a human saliva sample indicative of a SARS-CoV-2 viral infection in a subject, the kit comprising:
a solid support in fluid-flow communication with a saliva sample from the subject once the saliva sample is delivered to the solid support and on which a plurality of agents have been secured, directly or indirectly, which bind to one or more of the salivary biomarkers in the saliva sample, the salivary biomarkers being selected from the group consisting of Immunoglobulin G1 (IgG1), Immunoglobulin G3 (IgG3), Immunoglobulin G4 (IgG4), total Immunoglobulin G (IgG), Immunoglobulin M (IgM), Immunoglobulin A (IgA), Interleukin 2 (IL-2), Interleukin 6 (IL-6), Interleukin 10 (IL-10), Interferon gamma (IFN-γ), Hepatocyte growth factor (HGF), Colony stimulating factor 1 (CSF-1), Interleukin 18 (IL-18), and D-dimer; and
labeled antibodies specific for said selected salivary biomarkers,
wherein each agent binds to a different single salivary biomarker in the saliva sample, the kit detects the labeled antibodies on the bound salivary biomarkers, and the kit provides an indication if the concentration of the bound and labeled salivary biomarkers in the saliva sample is at or above a reference value correlated with a SARS-CoV-2 viral infection, and
wherein
the IgG1 reference value is about 39.6 mg/dl;
the IgG3 reference value is about 120 ng/ml;
the IgG4 reference value is about 23 mg/l;
the total IgG reference value is about 10.4 mg/dl;
the IgM reference value is about 120 mg/dl;
the IgA reference value is about 300 mg/dl;
the IL-2 reference value is about 5.6 pg/ml;
the IL-6 reference value is about 15 pg/ml;
the IL-10 reference value is about 11 pg/ml;
the IFN-γ reference value is about 200 ng/ml;
the HGF reference value is about 1.9 ng/ml;
the CSF-1 reference value is about 1200 pg/ml;
the IL-18 reference value is about 134 pg/ml; and
the D-dimer reference value is about 450 ng/ml.

2. The kit of claim 1 wherein the agent is a plurality of antibodies which bind with a specific one of the salivary biomarkers and the antibodies are secured to the solid support to thereby immobilize the bound salivary biomarkers on the solid support.

3. The kit of claim 2 wherein the labeled antibodies which bind with a specific one of the salivary biomarkers enable formation of a visible complex secured to the solid support if the one or more salivary biomarker is present in the saliva sample.

4. The kit of claim 3 wherein the antibodies secured to the solid support and the labeled antibodies bind with a specific one of the salivary biomarkers in the combination of salivary biomarkers selected from the group consisting of:
(a) IgG3 and IL-6;
(b) IgG3 and IgM;
(c) IgM and IL-6;
(d) IgG3 and CSF-1;
(e) IgG3 and HGF;
(f) IgG3 and D-dimer;
(g) IL-6 and CSF-1;
(h) CSF-1 and HGF; and
(i) HGF and D-dimer.

5. The kit of claim 4 wherein the antibodies secured to the solid support and the labeled antibodies bind with a specific one of the salivary biomarkers in the combination of salivary biomarkers selected from the group consisting of IgG3 and IgM and also with IL-6.

6. The kit of claim 5 wherein the kit is of a lateral flow immunochromatographic assay (LFA) type having one LFA lateral flow test strip with antibodies which bind with IgG3 secured thereto and a second LFA lateral flow test strip with antibodies which bind with IgM and with antibodies which bind with IL-6 secured thereto.

7. The kit of claim 6 wherein a visible indication is provided on the test strip if a reference value or amount of IgG3 in the saliva sample is about 120 ng/ml to about 200 ng/ml, a reference value or amount of IgM in the saliva sample is about 120 mg/dl to about 140 mg/dl, and a reference value or amount of IL-6 in the saliva sample is about 15 pg/ml to about 60 pg/ml.

8. The kit of claim 7 wherein the visible indication is provided on the test strip if the reference value or amount of IgG3 in the saliva sample is about 130 ng/ml to about 180 ng/ml, the reference value or amount of IgM in the saliva sample is about 123 mg/dl to about 130 mg/dl, and the reference value or amount of IL-6 in the saliva sample is about 20 pg/ml to about 40 pg/ml.

9. The kit of claim 8 further comprising instructions describing how to use the kit and interpret each visible indication.

10-20. (canceled)

21. A system for detecting biomarkers indicative of SARS-CoV-2 virus in a saliva sample obtained from a subject to determine whether the subject is infected with the virus, the system comprising:
(a) at least one binding agent specific to one or more biomarker, the one or more biomarker being selected from the group consisting of Immunoglobulin G1 (IgG1), Immunoglobulin G3 (IgG3), Immunoglobulin G4 (IgG4), total Immunoglobulin G (IgG), Immunoglobulin M (IgM), Immunoglobulin A (IgA), Interleukin 2 (IL-2), Interleukin 6 (IL-6), Interleukin 10 (IL-10), Interferon gamma (IFN-7), Hepatocyte growth factor (HGF), Colony stimulating factor 1 (CSF-1), Interleukin 18 (IL-18), and D-dimer;
(b) a measurable label that indicates a proportional reaction based on the amount of biomarker present in the saliva sample; and
(c) a measurement device operable to utilize the label to provide a qualitative and/or quantitative measure of the one or more biomarker indicative of whether the subject is infected with the SARS-CoV-2 virus.

22. The system of claim 21 wherein the at least one binding agent comprises antibodies, each antibody being specific for a biomarker.

23. The system of claim 22 further comprising a solid support to which the antibodies are attached, the solid support being selected from the group consisting of an enzyme-linked immunosorbent assay (ELISA) solid support and at least one lateral flow immunochromatographic assay (LFA) strip.

24. The system of claim 23 wherein the antibodies are capable of binding with a specific one of the biomarkers in the combination of biomarkers selected from the group consisting of:
(a) IgG3 and IL-6;
(b) IgG3 and IgM;
(c) IgM and IL-6;
(d) IgG3 and CSF-1;
(e) IgG3 and HGF;
(f) IgG3 and D-dimer;
(g) IL-6 and CSF-1;
(h) CSF-1 and HGF; and
(i) HGF and D-dimer.

25. The system of claim 24 wherein the antibodies are capable of separately binding to one of IgG3, IgM, and IL-6.

26. The system of claim 25 wherein the at least one LFA strip comprises a pair of LFA strips positioned and arranged to be concurrently contacted by the saliva sample and one of the pair of LFA strips includes antibodies specific to IgG3 and the other of the pair of LFA strips includes antibodies specific to IgM and IL-6.

27. The system of claim 26 wherein the pair of LFA strips produce a visible indication if the amount of IgG3 in the saliva sample is above about 130 ng/ml, if the amount of IL-6 in the saliva sample is above about 40 pg/ml, and/or if the amount of IgM in the saliva sample is above about 123 mg/dl.

28. The system of claim 27 wherein the measurement device provides a visual indication of the measurable label.

29. The system of claim 28 wherein the visual indication is a fluorescent indication.

30. A method of treating a patient afflicted with COVID-19 comprising:
(a) measuring the level of one or more biomarker selected from the group consisting of IgG1, IgG3, IgG4, Total IgG, IgM, IgA, IL-2, IL-6, IL-10, IFN-γ, HGF, CSF-1, IL-18, and D-dimer in a first saliva sample taken from the patient at a first time point;
(b) administering the patient with an antiviral drug or inhibitor for a treatment period;
(c) measuring the level of the one or more biomarker selected from the group consisting of IgG1, IgG3, IgG4, Total IgG, IgM, IgA, IL-2, IL-6, IL-10, IFN-γ, HGF, CSF-1, IL-18, and D-dimer in a second saliva sample at a subsequent second time point following the administration; and
(d) comparing the level of the one or more of IgG1, IgG3, IgG4, Total IgG, IgM, IgA, IL-2, IL-6, IL-10, IFN-γ, HGF, CSF-1, IL-18, and D-dimer biomarker at the first time point and the second time point,
wherein a change in the level of the one or more of IgG1, IgG3, IgG4, Total IgG, IgM, IgA, IL-2, IL-6, IL-10, IFN-γ, HGF, CSF-1, IL-18, and D-dimer biomarker at the second time point compared to the first time point indicates that the patient is responsive to treatment with the antiviral drug or inhibitor.

31. A ready-to-use lateral flow immunochromatographic assay (LFA) kit for detecting salivary biomarkers in a human saliva sample indicative of a SARS-CoV-2 viral infection in a subject, the kit comprising:
a first lateral flow test strip which receives the saliva sample, the first strip having a region with antibodies secured thereto which bind with salivary IgG3;
a second lateral flow test strip which also receives the saliva sample, the second strip having a first region with antibodies secured thereto which bind with salivary IgM and a second region with antibodies secured thereto which bind with salivary IL-6; and
labeled antibodies specific for IgG3 associated with the first strip and labeled antibodies specific for IgM and IL-6 associated with the second strip,
wherein formation of a detectable complex of salivary IgG and labeled antibodies at the region of the first strip, and formation of a detectable complex of salivary IgM and labeled antibodies and formation of a detectable complex of salivary IL-6 at the respective first and second regions of the second strip is indicative of a SARS-CoV-2 viral infection in the subject.

32. The kit of claim 31 wherein a visible indication is provided in the region of the first strip if an amount of salivary IgG3 in the saliva sample is at or above about 120 ng/ml, a visible indication is provided in the first region of the second strip if an amount of salivary IgM in the saliva sample is at or above about 120 mg/dl, and a visible indication is provided in the second region of the second strip if an amount of salivary IL-6 in the saliva sample is at or above about 15 pg/ml.

33. The kit of claim 32 wherein a visible indication is provided in the region of the first strip if the amount of salivary IgG3 in the saliva sample is within the range of about 120 ng/ml to about 200 ng/ml, a visible indication is provided in the first region of the second strip if the amount of salivary IgM in the saliva sample is within a range of about 120 mg/dl to about 140 mg/dl, and a visible indication is provided in the second region of the second strip if the amount of salivary IL-6 in the saliva sample is in a range of about 15 pg/ml to about 60 pg/ml.

34. The kit of claim 32 further including a cassette with a window, the first and second strips are housed within the cassette, and the visible indications are viewable through the window.

35. The kit of claim 32 further including a measurement device operable to utilize each indication to provide a qualitative and/or quantitative measure of the amount of salivary IgG, IgM, and IL-6 biomarker in the saliva sample, whereby said measure is utilized to determine whether the subject has the SARS-CoV-2 viral infection.
